SEQUENCE LISTING (3 sequences)

SEQ ID NO: 1 - DNA, 170 bp, Artificial Sequence
  MADM construct intron including loxP site
gttggtatcaaagatctataacttcgtatagcatacattatacgaagttatggttacaagacaggtttaaggagaccaatagaaactgggcatgtggagacagagaagactcttgggtttctgataggcactgactctctctgcctattggtctattttcccacccttag

SEQ ID NO: 2 - DNA, 5087 bp, Artificial Sequence
  Chromosome 11 5′ targeting arm
ggactagaatagttctgtgcttcaggactccatatgaaaccctgcactaaatatcccaaaggctcttgctttagagaaactccagaaggaattttgttcatgagttaggccctgtctcatcctttataaaattcattataaaattatataatttcattataaaataaacaagaacaaaggatccttaatggtaagttctagtaaggtgtacgataattgagttttcatgtatgtaattttaaaagaaaaatacaaggaacaagtggaatcataggttctgaggtctctgaaacatctcttccccattactaaggtatatttccagccatgctcttccctcttcttagccctatttcagggttgagaaacatgagaacacacacaggcccattttacagggtggcaaagatggattgaagttcttgggacaatgactacttgcccctttgtgttctcttgtagtagctataggtcagaacattagaattacctctgcccaagtaaacaaaatgggaactctttaccctgttgaagatggcttcacctggtaaagctaggcttaagagcgagtgctgcatagcccagtggtcaagtgctccaaagcatatgtaagaccctaggcttaattcccagtgtcctgaaaagataaagcactggcttagtgttaggcaaaggcacttgagtgtgctgatctctgcctcttctctgcagctttctccttccttcacccctacctcagtgatccggaagatgtatgaaagcagagagaaaaccaaggaggaaatggcacctggaatggtggttcctggtgatggtaaagaggatactcagaagaccagcgaaggtactgctgcttacctgtgtcagaacataggcatgagtgactgcagtgtcagggagatgggttagtgagctcaaacttgctcacttgacattgtggccagccggggtaacgatgtgctttagcttatgattcttaccatctttcaagttgaataacaaactcatttagcatattgatatagctcaattctttttttaacgtatttgtgtatttgcctgtggtatgtctgcatcatgtgtatccagtgcccaaggaggccagaagagggcatcagaacctctagaattgtgagccacatgtgggtgctgggaagaggtcctcttcaagagtatccaggtagatagggggatagatgcagggttgttgttgtttctgttacaggttgtttgttttatttgcaacaggatcttactgtgtagctgtggctaacctcaaactcacagagatctactggtctctgcctcccaagtcttaggattaaaggtgcctcaactacatgcctggcaacttgtgctttttgttttgtaacactatgtcagaggtttttgagttattcctttatgcaccgggaatgcagctcaattgtactaagtagtagaatacttgccagcatgtacaagaccatgggttcatgttcactccccaagaaaggaaatgacaacatcaaaaataattatttcagcaaagaatttgaagaccttggcatctatggactggagagatagatcagggcactgtttgctcttccagaggacctgggttcaattgccagcacccacatgatagctcaccttttgtgacttcagccctaggtgacctagcaccctcccctggcctctgtgagcactgaactcgacaggcacacagacatacatacaggcaaaacaccatccatatacatcataatataagaaaataaggttataaataaaatgtgggcatctattcctagctcaaggcctaactactctatgaccataactcaactccggggcttatggagagtaccagctgtgcatctgtctacatttaaagcaaatagatgactttccatgaaagcatctaacatggttaagaggaaaaaagagccggcatggtggcacacgcctttaatcccagcacttgggaagcataggcaggtggatttctgagttcgaggccagcctggtctacaaagtgagttccaggacagccagggctatacagagaaaccatgtcttgaaaaaccaaaaaaaaaaaaaaggaaaaaagaattgtcagaatttttaaaagtaagatttgctgggttgttgggaattgaaataaattgataaaggatatcaaaatgtcctgtaaatgaatccttacagagaacctgctgtcatccaatcccatacccaatactgatcaagactcttctacgacaaatcctaagctttcaacattacagcggtcttcatgttccaccccactctcccagaccagccgttataccaaagaacaagattatcgacctaaaacagcaggaaggaaaacacctaccttggcatccccagtcccaggaacaccttttctccgccctacccaccaagttccccttgtccctcatgttcctattgttcgacctgcacaccagcttcacccaggattagtccaaaggttgatagcccaaggggtacatccacagcatcttccaagtttgctccaagctggtgagtttctgactgggtgtgggcattggttattggtcgtagtataaaaattgctgccactaattaaatgttacattatactaagtattccaaaaagatcttactagatttaaattcttctacctaagtagtgagtccaatccagtctcacttccttagctcacaaaaagattaggaaacctgccagacagtggtaatacacgcctttaatccctgcactcaggaagcagaggcaggtggatctctttgagttcaaggatagccagggtacactaaggaaccctggctgtggcgttcatttacaattgtgtctgtgtatgcatattcatgtaccctggtacatgtatggtggcaagtgttcaccacatccacctccttgtaagtatccaaatcaacaaaagtagtaaactttgacatttttaggaacatctacaactcatgagtttcttctttctccccatcaggtgtgcttcctcctgggatagacatggctcctttacaggggctatctggcccactcctgggtcaacccttgtaccctttggtctctgctgctagtcaccctctcctaaaccctcgtcctgggacccctctgcatttggcggtgatgcagcagcagctacagcgctcaggtaaggtttagaaataggccaggttagctgatcacactggaaagttctgagatttccaggagcaatagttatcactgcccctttgtttcttaggtgtcactagaaaaacaggtgaagtgaactgacctgtgcagtctcagggattattactactgcaaggacagtcggagccagccatgctctagctctgtttttcacttcttataagcctgagaatttttgctgagatgtgaacatgtcagccttggtgggctttcttacgttgcattttcttacgttgcattttgcactttaggaagcctttggttaaatcctctggtagacttcccatcccccaaagtaacaaaccattcaagcaaagtaggtcagaaaatattgtcaatactgagaacaggacttgggagtgagtatccaagggtggtcttagtagacttgtggggttctgtgggtgaggtgtagagtagagctactttgctgcactgacactcttctctccatagttctgcatcctccaggctctagttcccaggcagcagctatcagcgttcagactcctcagaatgtacccagccggtcgggcatgccccacatgcactcccagctggagcatcgtaccagccaaaggagcagctcccctgtgggccttgccaaatggtttggctcagatgtgctacagcagcctctgccctccatgcccaccaaagtcatcagtgtagatgaactggaatatagacagtgaagagggcaggctggctcacccatacctggacctgtggtgacaccctggtcatgactctcattccctctttgtaatgggcttttacattggagcacactatgtgaagatgtttaggggatccacatacctaccatgatctacattatgacagaaggctgttaaatcgaatgaacctacatggttcaaatacaagggatacaagattgtcagtcctggaagtctttcttttataaaatatgtgaatgaagtgttggtgtcttctagaggtgacacctaagggttctgaaaaaataaaatgtatagacccttatgtacagacctgtgtataaacttttgtacatacaaatagggtagctttttttgaacttatacatacagctgtacataaagtaactatcagttaggcttgtgtcaactgtttggatttttttcacttgaatatttgggactttttcttttggtttattaaaagttacatatgccacgtgtgtgaacgatatggctggtactgtgtttatttcttccatgaactaagacagtctaaatgagttcctttcacgttttaattttaccttaggacttctggaatttcttctgcacataaagttctgatagcattagtttaagctggactaaccctgaaagtagcttgtggcaagtatcaaggaatcaatattatactctacaaaatcaaagtttacagagaagtcatatagtaatttttctgaaatttactggcacaatgttaatccagcctgactccaactaattaatggtcacattaatttaagtctttcccttgcctctgctgcattagtttctctcaaaattgttaacttacaacttgaagtctggtattataaattgaatgtaaagcattctgaaagatactatactgattgcaggtttttcagtcaggttcaagctaatttgaccagtcattggattaattatggatctggggccataaatgctattttaattccactatagagattaaaataagccattctccatttcataatattctattggactttgactgcaggggcctccaagtcttgacagtagattataatccttcagctgcccactctactggaggaggacaaactggtcacttttcagcaaaacctggctgtggatcagggcagtctggtacttccaagctcattagatgc

SEQ ID NO: 3 - DNA, 2938 bp, Artificial Sequence
  Chromosome 11 3′ targeting arm
catcatgctctcactgcctcctcagcttcaagaggaatctggaaaaagcagtcccactggtcaggaaaggaacactagtgcacttatcctgggtgtctgctgagccaacagtggtagtaaggtaagggcaggatgtgtcaaactgccaatagagaactacttactcttcaggctgaagctgatggaacaggtaacaaaggcaaacactaatcatgatcagcaagatgaagcagaaagggaacaaggggatattaaatgtgtatagacacgctagagagatggctcagcagttaagagaactagctggtctttcagaggtcctgagatcaattttagacacccacatggtggctcatgaccatctatctataaatggatctgattttcatgtctggcagtgtacagaagctaactgaagaaaggtggaagacccacaagagttcaagataagccctatatagtgaagttcaaggcaagctttttctacctgaaacttagtctcaaaaaaaaatgaatacgtaaacagtcttccaggggataagaaccttacagaaaaagcagaaatgcctggggcactggattaccgatgtaatcaaattcagtccttgaattgaacacaggattgcctagagcaaggccagccagagattcatctcagagggagaaaggtgtctttggagcaattttgtggtaatctagtatgtatcacataagtttagacgcatttgggactggaaagatgtgaacaaagcaccctatggctcacatctgtcattaactctagttccagtgcacctgacaccgtcttctggcctctgcagtgaccaagcacatgggtagtatgtagacatatacataagcaaaacacacatcattaaaaagtgacatttcccaaaggaagctgaagaaccagttcttgagaagatagtagaaatcagaaggggaaatagtagacatacagagggactgaccaggttgtgtcacctttataggctaggctaatggatgatcgacactagcgctctttgtgaaggacacacaaatgagacatagtttataggactaaacacacttctaagcaatttaatgagacttaagaccctgtctctagcaaatactctggatgatattcagctcaaggctcttgtcagacatgtttccattttcaaggtgagctaactggccaaaactgccaacaacctgtagtgaatagagaagatgagaaaatctggattctcaaatgacctaatgaaagccactggagcgccatatggtttctgtgaaaatgccttttcaatcattaacctcttaaatgagtgttagcatcctaactaatgagtggtgcagaatagtgggtctgcttagcttaactaaggccaagaaaacaaaacaggaaattcattccatgtcatgagactcatactacgaggttcccttagacctcaggagaaaaagtctttggctgtaagaacacacctcagtggatgtggtagactatgcctttactcttttttttttttttttttttttttgggttttttcgagacagggtttctctgtgtagccttggctgtcctggaactcactttgttgaccaggctggcctcgaactcagagatccgcctgcctctgcctctcaagtgctagaattaaaagtgtgcgccaccactcccagctatatagactatgcctttaatcaggacacttgggaggcagggggatctcaaattcaattccagacagatgactgacaaacacacacatacaccattcgtcttttctttttttcttttttcttttgaaaacaggtttctctatgcagccctggctttcctagaattcaatctatagacccagctggcctcaaacacagaacctcttgcctctgcctcctcaacactatgactaaaggtgtgaaccaccaccaccaccacccacctggccaaagaatgaattgagtgaataagtacgagtgcaactctccagcagcctgaggcaagaaagcttaacagttgaacctgagaccctacttcgtttgtgttgtacatatcttctcataatgaactaggcagcctagtctctctaccaaatacaccatgcacttacacctcctcaaaccaactgcaacagctacacacatttccatggcttgcctgtaataataaattcacaaacttcctctaagtaacttttagtggaaatctaccaggttatgcctatttgtgattgggtgcacagatgactagatgagatgtaatttgggaagtgacatgcaataggaaaaatgaatatataacctataaatagaaaacaccaaggcaagcgtggtgactcacacctttaattagcacttgggaggcagaagtcgcatcatctcttaggtcaaggccaatctagttccagaccagcaaggacaacagaaaccttgccttgatataaacattttaaaactttaaaaatagagaagcccaaactctgctcattggtaatctacccttcctttctttagacaccttgagaagctaggctacatagctttgggtccttacgtacccccactcactcagcctgcagtcaatctctgatagtgtttcctgccataagctacactactgttttgctccaaaccttacttctagtacaaatccatgtgaggtcttcgtttcaatttgggggggaagaggccagaacacttgccgcagatcccaacaacgggcagcacattcccagctagtgtatctgatacaaactctatcttcattcagaaccatcaagtctaagccccaagagcaaaggtaaattcatgtatgtttggctggagaaagcatttcaagtcaaggtgagccttgaactcctaacactcttgcctctaccttccaaatgccaatataggtatttgtcacaccagcttctcaaatcctgttgggctagcaactgg

(19) United States
(12) Patent Application Publication
(10) Pub. No.: US 2011/0035815 A1
(43) Pub. Date: Feb. 10, 2011

(54) Mosaic knockout mouse tumor models and methods or use

(75) Inventors: Hui Zong, Eugene, OR (US); Chong Liu, Eugene, OR (US)

Correspondence Address: KLARQUIST SPARKMAN, LLP, 121 SW SALMON STREET, SUITE 1600, PORTLAND, OR, 97204, US

(73) Assignees: The State of Oregon Acting by and through the State Board of Higher Education on behalf of the; University of Oregon

(21) Appl. No.: 12/806,302

(22) Filed: Aug. 9, 2010

Related U.S. Application Data
(60) Provisional application No. 61/273,903, filed on Aug. 10, 2009.

(51) Int. Cl. G01N 33/48; A01K 67/027; C12Q 1/02; C12Q 1/68

(52) U.S. Cl. 800/3; 800/18; 435/29; 435/6

(57) ABSTRACT

Disclosed herein are transgenic mouse tumor models. A portion of the cells of the disclosed transgenic mice undergo directed somatic recombination and the mouse is therefore a mosaic for homozygous knockout of p53, NF1, or both p53 and NF1 genes. The homozygous null cells resulting from the somatic recombination also express a detectable fluorescent protein, and the resulting sibling cells, which are wild type for p53, NF1, or both p53 and NF1, express a different detectable fluorescent protein. Also disclosed herein are methods of identifying compounds for treating or preventing a tumor, using the disclosed transgenic mice.

### CROSS REFERENCE TO RELATED APPLICATION

This claims the benefit of U.S. Provisional Application No. 61/273,903, filed Aug. 10, 2009, which is incorporated herein in its entirety.

### FIELD

This disclosure relates to a transgenic mouse tumor model wherein the transgenic mouse is mosaic for homozygous knockout of p53, NF1, or both p53 and NF1 genes. The homozygous null cells express a detectable fluorescent protein and sibling cells, which are wild type, express a different detectable fluorescent protein. The disclosure also relates to use of the transgenic mice in methods for identifying compounds for treating or preventing a tumor.

### BACKGROUND

Brain tumors account for about 1-2% of adult tumors, with tumors arising from glial cells (gliomas) being the most common type of brain tumor (about 60%). Gliomas can arise from astrocytes (astrocytoma), oligodendrocytes (oligodendroglioma), or ependymal cells (ependymoma). Gliomas are divided into four grades; Grades I and II are considered low grade and Grades III and IV are considered high grade. Glioblastoma multiforme, also known as grade IV astrocytoma, is the most common form of glioma. Standard treatment for glioma includes surgical resection. High grade glioma is also treated with radiation therapy and chemotherapy (such as temozolomide).

Glioma is difficult to detect, since patients are often asymptomatic until tumors reach late stages. Once detected, it often progresses rapidly and generally leads to patient mortality within one year. One of the reasons contributing to the high rate of mortality is the diffuse nature of glioma cells. The tumor cells infiltrate into many areas in the brain, making complete resection impossible. Patients mostly die of tumor recurrence shortly after surgery. Human genetic studies revealed that p53 and NF1 are among the most frequently mutated tumor suppressor genes in gliomas (Parsons et al., Science 321:1807-1812, 2008; Reilly and Jacks, Semin. Cancer Biol. 11:177-191, 2001).

### SUMMARY

Disclosed herein are transgenic mouse tumor models. In one embodiment, a portion of the cells of a disclosed transgenic mouse undergo directed somatic recombination and the mouse is therefore a mosaic for homozygous knockout of both p53 and NF1 genes (p53-NF1 MADM mouse). In another embodiment, a portion of the cells in a disclosed transgenic mouse undergo directed somatic recombination and the mouse is a mosaic for homozygous knockout of the p53 gene (p53 MADM mouse). In a further embodiment, a portion of the cells in a disclosed transgenic mouse undergo directed somatic recombination and the mouse is a mosaic for homozygous knockout of the NF1 gene (NF1 MADM mouse). The homozygous null cells resulting from the somatic recombination also express a detectable fluorescent protein (such as green fluorescent protein (GFP)), and the resulting sibling cells, which are wild type for both p53 and NF1 (p53-NF1 MADM mouse), wild type for p53 (p53 MADM mouse), or wild type for NF1 (NF1 MADM mouse) express a different detectable fluorescent protein (such as a red fluorescent protein (RFP)). The mosaic nature of the mouse for cells that are homozygous null (for example, for p53, NF1, or both p53 and NF1) more closely models the loss of heterozygosity (LOH) of tumor suppressor genes (TSG) that frequently contributes to tumor development than conventional mouse knockout models (where all of the cells in the mouse or in a particular tissue are homozygous null). In addition, the differential detectable labeling of homozygous null and wild type cells allows the development and fate of tumor cells and normal cells to be followed either in vitro or in vivo.

In particular examples, the disclosed transgenic mice have a genome that includes a first nucleic acid molecule including a first promoter operably linked to a nucleic acid molecule encoding an N-terminal portion of a GFP and a C-terminal portion of a red fluorescent protein (RFP; such as a tdTomato (tdT) fluorescent protein), wherein the N-terminal portion of the GFP and the C-terminal portion of the RFP are separated by an intron (such as a β-globin intron) including a first loxP site, and wherein the first nucleic acid molecule is present at a locus of a first chromosome 11 of a chromosome 11 pair, and a second nucleic acid molecule that includes a second promoter operably linked to a nucleic acid molecule encoding an N-terminal portion of the RFP and a C-terminal portion of the GFP, wherein the N-terminal portion of the RFP and the C-terminal portion of the GFP are separated by an intron (such as a β-globin intron) including a second loxP site, and wherein the second nucleic acid molecule is present at the homologous (equivalent) locus of the second chromosome 11 of the chromosome 11 pair. The genome of the transgenic mice also include a mutated gene selected from a heterozygous null mutation in a p53 gene, a heterozygous NF1 gene including two loxP sites (“foxed” NF1), and both a heterozygous null mutation in the p53 gene and a heterozygous floxed NF1 gene; the mutated gene(s) were originally introduced in the mouse genome by homologous recombination and introduced on the MADM-bearing chromosome by standard breeding techniques. In some examples, the mutated gene(s) are present in cis on the second chromosome 11 of the chromosome 11 pair. In other examples, the mutated gene(s) are present in cis on the first chromosome of the chromosome 11 pair.

In a particular embodiment, a disclosed transgenic mouse has a genome that includes a first nucleic acid molecule including a first promoter operably linked to a nucleic acid molecule encoding an N-terminal portion of a GFP and a C-terminal portion of a red fluorescent protein (RFP; such as a tdTomato (tdT) fluorescent protein), wherein the N-terminal portion of the GFP and the C-terminal portion of the RFP are separated by an intron (such as a β-globin intron) including a first loxP site, and wherein the first nucleic acid molecule is present at a locus of a first chromosome 11 of a chromosome 11 pair, and a second nucleic acid molecule that includes a second promoter operably linked to a nucleic acid molecule encoding an N-terminal portion of the RFP and a C-terminal portion of the GFP, wherein the N-terminal portion of the RFP and the C-terminal portion of the GFP are separated by an intron (such as a β-globin intron) including a second loxP site, and wherein the second nucleic acid molecule is present at the homologous (equivalent) locus of the second chromosome 11 of the chromosome 11 pair. In particular examples, the locus of chromosome 11 which includes the first and second nucleic acid molecules is between the Eif4enif1 and Drg1 genes. The genome of the transgenic mouse also includes a heterozygous null mutation in the p53 gene and a heterozygous NF1 gene including two loxP sites (“floxed” NF1); both of which were originally introduced in the mouse genome by homologous recombination and introduced on the MADM-bearing chromosome by standard breeding techniques. In some examples, the p53 null mutation and the floxed NF1 gene are present in cis on the second chromosome 11 of the chromosome 11 pair. In other examples, the p53 null mutation and the floxed NF1 gene are present in cis on the first chromosome of the chromosome 11 pair.

In another particular embodiment, a disclosed transgenic mouse has a genome that includes a first nucleic acid molecule including a first promoter operably linked to a nucleic acid molecule encoding an N-terminal portion of a GFP and a C-terminal portion of an RFP (such as tdT fluorescent protein), wherein the N-terminal portion of the GFP and the C-terminal portion of the RFP are separated by an intron (such as a β-globin intron) including a first loxP site, and wherein the first nucleic acid molecule is present at a locus of a first chromosome 11 of a chromosome 11 pair, and a second nucleic acid molecule that includes a second promoter operably linked to a nucleic acid molecule encoding an N-terminal portion of the RFP and a C-terminal portion of the GFP, wherein the N-terminal portion of the RFP and the C-terminal portion of the GFP are separated by an intron (such as a β-globin intron) including a second loxP site, and wherein the second nucleic acid molecule is present at the homologous locus of the second chromosome 11 of the chromosome 11 pair. In particular examples, the locus of chromosome 11 which includes the first and second nucleic acid molecules is between the Eif4enif1 and Drg1 genes. The genome of the transgenic mouse also includes a heterozygous null mutation in the p53 gene which was originally introduced in the mouse genome by homologous recombination and introduced on the MADM-bearing chromosome by standard breeding techniques. In some examples, the p53 null mutation is present in cis on the second chromosome 11 of the chromosome 11 pair. In other examples, the p53 null mutation is present in cis on the first chromosome of the chromosome 11 pair.

In a further embodiment, a disclosed transgenic mouse has a genome that includes a first nucleic acid molecule including a first promoter operably linked to a nucleic acid molecule encoding an N-terminal portion of a GFP and a C-terminal portion of an RFP (such as tdT fluorescent protein), wherein the N-terminal portion of the GFP and the C-terminal portion of the tdT protein are separated by an intron (such as a β-globin intron) including a first loxP site, and wherein the first nucleic acid molecule is present at a locus of a first chromosome 11 of a chromosome 11 pair, and a second nucleic acid molecule that includes a second promoter operably linked to a nucleic acid molecule encoding an N-terminal portion of the RFP and a C-terminal portion of the GFP, wherein the N-terminal portion of the RFP and the C-terminal portion of the GFP are separated by an intron (such as a β-globin intron) including a second loxP site, and wherein the second nucleic acid molecule is present at the homologous locus of the second chromosome 11 of the chromosome 11 pair. In particular examples, the locus of chromosome 11 which includes the first and second nucleic acid molecules is between the Eif4enif1 and Drg1 genes. The genome of the transgenic mouse also includes a heterozygous NF1 gene including two loxP sites (“foxed” NF1) which was originally introduced in the mouse genome by homologous recombination and introduced on the MADM-bearing chromosome by standard breeding techniques. In some examples, the floxed NF1 gene is present in cis on the second chromosome 11 of the chromosome 11 pair. In other examples, the foxed NF1 gene is present in cis on the first chromosome of the chromosome 11 pair.

The transgenic mouse (such as a p53 MADM mouse, NF1 MADM mouse, or p53-NF1 MADM mouse) further includes a nucleic acid molecule encoding a Cre recombinase operably linked to a promoter, such as a tissue-specific, cell-type specific, temporally-specific, or inducible promoter. In one particular example, the transgenic mouse includes a nucleic acid encoding a Cre recombinase operably linked to a promoter that is active in neural stem cells and/or glial precursor cells (such as a human glial fibrillary acidic protein (hGFAP) promoter or a Nestin promoter).

Also disclosed herein are methods of identifying compounds for treating or preventing a tumor (such as glioma), using the disclosed MADM transgenic mice. In one embodiment, the method includes culturing at least one first cell from a disclosed MADM transgenic mouse that is homozygous null for p53, NF1, or both p53 and NF1 and which expresses a fluorescent protein (such as GFP), contacting the cell with at least one test compound, determining a phenotype of the cell or a population of cells (such as cell number, cell proliferation, cell cycle stage, cell death, tumor sphere formation or characteristics, or cell differentiation), and selecting a compound that alters (for example, decreases or increases) the phenotype as compared to a control. In a particular example, the method further includes co-culturing the at least one first cell with at least one second cell that is wild type and which expresses a different fluorescent protein than the first cell (such as tdT) and determining a ratio of the number of first to second cells (such as the ratio of the number of green cells to the number of red cells).

In another embodiment, the screening method includes administering at least one test compound to a MADM transgenic mouse of the disclosure, determining a phenotype of the transgenic mouse (such as the number of fluorescent protein-expressing cells, tumor size, tumor grade, tumor number, tumor metastasis, tumor recurrence, gene expression, morbidity, or mortality), and selecting a compound that alters (for example, decreases) the phenotype as compared to a control (such as a transgenic mouse not administered the at least one test compound).

The foregoing and other features will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A is a schematic diagram illustrating the generation of green (GFP expressing) mutant cells and red (RFP expressing) sibling wild type cells utilizing the MADM system.

FIG. 1B is a schematic diagram illustrating the breeding scheme used to generate p53-NF1 MADM mice. Semicolon indicates that Cre is on a different chromosome than the MADM construct.

FIG. 2A is a bar graph showing quantification of the green cell to red cell (G/R) ratio in MADM-mutant brains from P5 to P60. Three slices for each mouse brain were analyzed, and in each slice 9 regions were examined. Error bars represent +/−SEM.

FIG. 2B is a bar graph showing the average number of all 5-bromo-2-deoxyuridine (BrdU) positive cells in p53-NF1 MADM mouse brain after normalized in a single confocal image area (0.1 mm²). Mice were administered BrdU 1.5 hours before sacrifice. Three slices for each mouse brain were analyzed, and in each slice 9 regions were examined. Error bars represent +/−SEM.

FIG. 3 is a pair of digital images of a brain section from a 4 month old p53-NF1 MADM mouse stained with hematoxylin and eosin showing key pathological features of malignant gliomas. The right panel is a higher magnification of the boxed region in the left panel. N, pseudopalisading necrosis; arrows, prominent blood vessels.

FIG. 4 is a digital image showing a sagittal brain section from a 4 month old p53-NF1 MADM mouse showing two malignant gliomas (dashed lines) consisting mostly of p53 and NF1 null mutant (green) cells.

FIG. 5 is a series of digital images showing expression of molecular markers in the tumor region of a p53-NF1 MADM mouse. Immunofluorescence staining shows the tumor region (visualized in MADM staining), from a Nestin-Cre induced tumor enhanced the expression of Ki67, Nestin, Sox2, Olig2, PDGFRα, NG2, CD9, and GFAP in the tumor, but not neurofilament light or medium chain (NF-L/M), NeuN, or CC-1. All staining derived from adjacent sections of the same tumor. Tumor boundary is demarcated by dashed line. T, tumor mass. Scale bars 200 except for the image for CD9, which is 50 μm.

FIG. 6A is a series of schematics showing models of possible phenotypes with over-expansion of mutant cells occurring in particular cell types. Dark cells are red (WT) and light cells are green (mutant).

FIG. 6B is a digital image showing brain areas defined for counting the G/R ratio of cells. The sagittally sectioned brain was subdivided into up to 21 regions, including most gray and white matter, but not the neurogenic region. Cells were counted in each region and the G/R ratio was calculated.

FIG. 6C is a bar graph showing the quantification of G/R ratio (+/−SEM) in neuron (N), astrocyte (As), oligodendrocyte (OLi) and oligodendrocyte precursor cell (OPC) populations in 2-month old p53-NF1 MADM-mutant mouse brains. This demonstrates that OPC is the predominant cell type in the over-expansion phenotype, suggesting that model 3 (FIG. 6A) most accurately reflects events in the p53-NF1 MADM mouse.

FIG. 6D is a pie chart showing quantification of BrdU+ cells (either PDGFRα+ or −) at the brain parenchyma following 7 days BrdU labeling.

FIG. 6E is a bar graph showing the percentage of BrdU, PDGFRα double positive cells in all mutant or heterozygous PDGFRα+ cells in FIG. 6D. Error bar represents +/−SD. *p<0.01, paired T-test.

FIG. 7 is a series of digital images showing expression of OPC markers in p53-NF1 MADM mouse. Identifiable double positive (indicated marker and Ki67) tumor cells are indicated with arrows. Scale bars, 20 μm.

FIG. 8A is a schematic illustrating the immunopanning method used to isolated PDGFRα+tumor cells. Sequential BSL-1 panning was used to deplete microglia. OPCs were purified by their adhesion to PDGFRα-coated dishes.

FIG. 8B is a schematic showing relative expression of the indicated genes from panned tumor cells, OPCs and embryonic NSCs. The same tumor samples were consistently labeled with the same names. Tumors T1 and T3 were induced by hGFAP-Cre. Tumor T2 was induced by Nestin-Cre. T1-2 was the secondary tumor from T1.

FIG. 8C is a schematic illustrating single sample gene set enrichment analysis (GSEA) of neural lineage gene sets of tumor cells from p53-NF1 MADM mice compared with four cell types (neuron (N), astrocyte (As), oligodendrocyte (OLIG) and oligodendrocyte precursor cell (OPC)). Only the transcriptome of OPCs showed significant similarity to the tumor cells.

FIG. 8D is a schematic illustrating singe sample GSEA activation scores of glioblastoma multiforme (GBM) subtypes compared to tumor cells from p53-NF1 MADM mice. The p53-NF1 MADM mouse tumor cells closely resemble the proneural subtype of human GBM.

FIG. 9A is a series of digital images showing GFP (left) and nestin (center) expression in tumor spheres and an overlay of the GFP and nestin images with DAPI staining (right).

FIG. 9B is a series of digital images showing GFP (left) and Sox2 (center) expression in tumor spheres and an overlay of the GFP and Sox2 images with DAPI staining (right).

FIG. 9C is a digital image of tumor cells showing GFAP expression following culture in the presence of 1% serum.

FIG. 9D is a digital image of tumor cells showing microtubule-associated protein 2 (MAP2) expression following culture in the presence of 1% serum.

FIG. 9E is a digital image of tumor cells showing oligodendrocyte marker 04 expression following culture in the presence of 1% serum.

FIG. 10 is a bar graph (left panel) showing tumor sphere formation of freshly isolated cells (100 lives cells/μl) from PDGFRα-immunopanning plates (P) or the supernatant (S). Cells were cultured with either EGF/FGF-2 or PDGF-AA. Each represents the average of 5 replicates. Error bars represent +/−SD. **p<0.001, t-test. The right panel shows the representative phase-contrast images from the P or S fractions.

### SEQUENCE LISTING

Any nucleic acid and amino acid sequences listed herein or in the accompanying sequence listing are shown using standard letter abbreviations for nucleotide bases, and three letter code for amino acids, as defined in 37 C.F.R. 1.822. In at least some cases, only one strand of each nucleic acid sequence is shown, but the complementary strand is understood as included by any reference to the displayed strand.

The Sequence Listing is submitted as an ASCII text file in the form of the file named Sequence_Listing.txt, which was created on Jul. 21, 2010, and is 11,462 bytes, which is incorporated by reference herein.

SEQ ID NO: 1 is the nucleic acid sequence of a β-globin intron including a loxP site.

SEQ ID NO: 2 is the nucleic acid sequence of a mouse chromosome 11 5′ targeting arm.

SEQ ID NO: 3 is the nucleic acid sequence of a mouse chromosome 11 3′ targeting arm.

### DETAILED DESCRIPTION

### I. Abbreviations

BrdU: 5-bromo-2-deoxyuridine

CNS: central nervous system

EGFP: enhanced green fluorescent protein

FACS: fluorescence activated cell sorting

GFAP: glial fibrillary acidic protein

GFP: green fluorescent protein

LOH: loss of heterozygosity

MADM: mosaic analysis with double marker

MAP2: microtubule associated protein 2

NF1: neurofibromatosis type 1

NSC: neural stem cell

OPC: oligodendrocyte precursor cell

PDGFRα: platelet-derived growth factor receptor alpha

RFP: red fluorescent protein

RT-PCR: reverse transcription-polymerase chain reaction

SVZ: subventricular zone

tdT: tandem dimer Tomato fluorescent protein

TSG: tumor suppressor gene

### II. Terms

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. Similarly, the word “or” is intended to include “and” unless the context clearly indicates otherwise. The term “comprises” means “includes.” It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. All publications, patent applications, patents, and other references mentioned herein are incorporated by reference in their entirety for all purposes. In case of conflict, the present specification, including explanations of terms, will control. In addition, the materials, methods, and examples are illustrative only and not intended to be limiting.

Mouse strains are named using the following nomenclature system. A “,” is used to indicate two genes on the same chromosome (in cis). A “I” is used to designate genes on separate homologous chromosomes (in trans). Finally, a “;” is used to separate genes that are not linked to the preceding chromosome.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); Robert A. Meyers (ed.), Molecular Biology and Biotechnology: a Comprehensive Desk Reference, published by VCH Publishers, Inc., 1995 (ISBN 1-56081-569-8); and King et al., A Dictionary of Genetics, 7^{th} edition, published by Oxford University Press, 2006 (ISBN 0-19-530761-0).

In order to facilitate review of the various embodiments of the disclosure, the following explanations of specific terms are provided:

Contacting: A state or condition of touching or being in immediate physical proximity, particularly direct physical association, for example both in solid form and/or in liquid form (for example, the placement of a test compound in contact with a cell).

Control: A “control” refers to a sample or standard used for comparison with an experimental or test sample. In some embodiments, the control is a mouse, such as a transgenic mouse disclosed herein to which a test compound has not been administered. In other embodiments, the control is a cell, such as a cell from a transgenic mouse disclosed herein, which has not been contacted with a test compound.

Cre recombinase (Cre): A member of the integrase family of recombinases from P1 bacteriophage that catalyzes site-specific recombination of DNA between loxP recognition sites. The loxP site is a 34 base pair sequence including two 13 base pair inverted repeats separated by an 8 base pair spacer. Recombination products depend on the number and orientation of the loxP sites. If two loxP sites are located on different DNA molecules (for example, in trans), translocation between the two molecules (for example, chromosomes) will occur. In other examples, DNA between two loxP sites in the same orientation will be excised and DNA between loxP sites in opposite orientations will be inverted with respect to its starting orientation. See, e.g. Nagy, Genesis 26:99-109, 2000. In a particular example, Cre catalyzes site-specific recombination between two loxP sites, each at an identical location on homologous chromosomes, for example mouse chromosome 11.

Fluorescent protein: One of a number of proteins that emit fluorescence of a particular wavelength (color) when excited by a different wavelength of light. Green fluorescent protein (GFP) was the first identified member of this family. GFPs are derived from marine organisms such as Aequorea victoria and Renilla reniformis, and emit green fluorescence when exposed to blue light. GFP includes both wild type GFP and derivatives of GFP, such as GFP with one or more mutations that improve characteristics of fluorescence, photostability, or folding of the protein (for example, enhanced GFP (EGFP); see, e.g., U.S. Pat. No. 6,172,188). Additional mutations in GFPs produce fluorescent proteins with different colored emissions. These include blue fluorescent proteins, cyan fluorescent proteins, and yellow fluorescent proteins.

Fluorescent proteins also include red fluorescent proteins (RFP), such as the tetrameric DsRed, derived from the coral Discosoma (see e.g., Matz et al., Nature Biotechnol. 17:969-973, 1999). RFPs also include variants of DsRed, such as variants that form monomers or dimers, or have altered emission spectra (ranging from yellow to red), including tdTomato (tdT), mCherry, mStrawberry, mOrange, and mBanana (see e.g., U.S. Pat. Publication 2005/0196768; Shaner et al., Nature Biotechnol. 22:1567-1572, 2004).

In particular examples, fluorescent proteins utilized herein include EGFP and tdTomato.

Glioma: A tumor that arises from glial cells. The particular type of glioma is defined based on the type of glial cell from which the tumor arises or is believed to arise. For example astrocytoma (including glioblastoma, for example glioblastoma multiforme) is believed to arise from astrocytes, oligodendroglioma is believed to arise from oligodendrocytes, and ependymoma is believed to arise from ependymal cells.

Operably linked: A first nucleic acid molecule is operably linked to a second nucleic acid molecule when the first nucleic acid molecule is placed in a functional relationship with the second nucleic acid molecule. For instance, a promoter is operably linked to a coding nucleic acid molecule if the promoter affects the transcription or expression of the coding nucleic acid molecule. Generally, operably linked nucleic acid molecules are contiguous and, where necessary to join two protein coding regions, in the same reading frame. If one or more introns are present, the operably linked nucleic acid molecules may not be contiguous.

Pharmaceutically acceptable carrier: The pharmaceutically acceptable carriers (vehicles) useful in this disclosure are conventional. Remington: The Science and Practice of Pharmacy, ed., Hendrickson, Lippincott Williams & Wilkins, Philadelphia, Pa., 21^{st} Edition (2005), describes compositions and formulations suitable for pharmaceutical delivery of one or more compounds or molecules, such as one or more test compounds and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Promoter: A nucleic acid molecule including an array of nucleic acid control sequences which direct transcription of a nucleic acid molecule. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor or a compound (such as tetracycline)).

Purified: The term purified does not require absolute purity; rather, it is intended as a relative term. Thus, for example, a purified cell preparation is one in which the specified cell or cell type is more enriched than it is in vivo. Preferably, a preparation of a specified cell or cell type is purified such that the cell or cell type represents at least 50% of the total cell content of the preparation. In some embodiments, a purified preparation contains at least 60%, at least 70%, at least 80%, at least 85%, at least 90%, at least 95%, or more of the specified cell type, such as a cell that is homozygous null for p53 and NF1 and which expresses GFP (or tdT).

Ratio: A quantitative relation between two amounts showing the number of times one amount contains or is contained within the other; a proportional relationship between two different numbers or quantities. A ratio can be calculated by dividing one number or value by another. In some examples, a ratio includes the proportion of cells homozygous null for p53 and NF1 and expressing a fluorescent protein (such as GFP) compared to cells wild type for p53 and NF1 and expressing a different fluorescent protein (such as tdT) in a sample (such as a tissue sample or a cell culture).

Somatic recombination: Recombination of genetic material by homologous crossing over in somatic cells, such as in cells in culture or in a living animal (for example, a mouse, such as a transgenic mouse). In one example, somatic recombination includes interchromosomal mitotic recombination, such as Cre-mediated recombination at loxP sites at homologous locations on each member of a chromosome pair.

Transgenic mouse: A mouse having at least one non-endogenous (heterologous) nucleic acid molecule present as an extrachromosomal element in a portion of its cells or stably integrated into its germline DNA (i.e., in the genomic sequence of some, most, or all of its cells). A heterologous nucleic acid molecule is introduced into the germ line of such transgenic mice by genetic manipulation of, for example, embryos or embryonic stem cells according to methods well known in the art. A “transgene” is meant to refer to such heterologous nucleic acid, such as a heterologous nucleic acid molecule in the form of an expression construct (such as for the production of a “knock-in” transgenic animal) or a heterologous nucleic acid molecule that upon insertion within or adjacent to a target gene results in a decrease in target gene expression (such as for production of a “knock-out” transgenic animal). A “knock-out,” or “null mutation,” of a gene means an alteration in the sequence of the gene that results in a decrease of function of the target gene, preferably such that target gene expression is undetectable or insignificant. Transgenic knock-out animals can comprise a heterozygous knock-out of a target gene, or a homozygous knock-out of a target gene. “Knock-outs” also include conditional knock-outs, where alteration of the target gene can occur upon, for example, exposure of the animal to a substance that promotes target gene alteration, introduction of an enzyme that promotes recombination at the target gene site (for example, Cre in the Cre-lox system), or other methods for directing the target gene alteration.

Treating: A therapeutic intervention that ameliorates a sign or symptom of a disease or pathological condition related to a disease (such as a tumor, for example, a glioma). Treatment can also induce remission or cure of a condition, such as a tumor. Reducing a sign or symptom associated with a tumor (such as a glioma) can be evidenced, for example, by a delayed onset of clinical symptoms of the disease, a reduction in severity of some or all clinical symptoms of the disease, a slower progression of the disease (for example by prolonging the life of a subject having a tumor), a reduction in the number of relapses or recurrences of the disease, an improvement in the overall health or well-being of the subject, or by other parameters well known in the art that are specific to the particular tumor.

Treatment includes preventing a disease, for example by inhibiting the full development of a disease, such as preventing development of a tumor (such as a metastasis or the development of a primary tumor) or a tumor recurrence. Prevention does not always require a total absence of a tumor.

Tumor: A neoplasm that may be either malignant or non-malignant (benign). Tumors of the same tissue type are tumors originating in a particular organ (such as brain, breast, lung, or colon) or cell type (such as glial cell, for example astrocyte or oligodendrocyte). In particular examples, a tumor includes a glioma (such as an astrocytoma or a glioblastoma).

Tumors include original (primary) tumors, recurrent tumors, and metastases (secondary tumors). A tumor recurrence is the return of a tumor, at the same site (for example, in the same organ or tissue) as the original (primary) tumor, after the tumor has been removed surgically, by drug or other treatment, or has otherwise disappeared. A metastasis is the spread of a tumor from one part of the body to another. Tumors formed from cells that have spread are called secondary tumors (or metastatic tumors) and contain cells that are like those in the original (primary) tumor. There can be a recurrence of either a primary tumor or a metastasis.

Tumor suppressor: A nucleic acid or the polypeptide it encodes, that in its wild type form has the ability to suppress, prevent, or decrease uncontrolled cell growth and or cell/division. Tumor suppressor genes (TSG) encode proteins that directly or indirectly inhibit progression through the cell cycle thereby inhibiting cell growth and/or cell division. When DNA damage is detected in a cell, tumor suppressors may prevent the cell from continuing to multiply until the damaged DNA is repaired. Alternatively, if the DNA cannot be repaired, they may signal the cell to undergo apoptosis (programmed cell death) in order prevent the damaged DNA from being passed on to the daughter cells. Tumor suppressors therefore play an important role in preventing the onset of uncontrolled cell growth, or neoplasia. Examples of tumor suppressors are p53, NF1, BRCA1, and NF2.

### III. MADM Mouse Tumor Model

One important event for human tumor initiation is the loss of heterozygosity (LOH) of tumor suppressor genes, which have been modeled in mice using gene knockouts. However, current mouse genetic models have difficulties in controlling gene inactivation in a small number of cells to mimic the sporadic nature of the LOH events typical of human tumors. These models also lack the resolution needed to address early tumor progression and tumor-niche interaction problems.

Disclosed herein are mouse tumor models generated using the MADM system (see U.S. Pat. No. 7,282,621; Zong et al., Cell, 121:479-492, 2005; Muzumdar et al., Proc. Natl. Acad. Sci., 104:4495-4500, 2007; all of which are incorporated herein by reference). In particular examples, the mouse is a tumor model which is a transgenic mouse mosaic for knockout of p53, NF1, or both p53 and NF1 genes (for example, in glial cells or precursors), in which cells homozygous null for p53, NF1, or both p53 and NF1 are differentially labeled as compared to cells wild type for these genes, and which transgenic mouse develops a tumor (such as glioma).

A. Mosaic Analysis with Double Markers (MADM)

Mosaic, as a genetic term, refers to a subject having cells of different genotypes. Subjects with a tumor are a genetic mosaic because the tumor cells contain gene mutations that are not present in non-tumor cells in the subject and therefore the tumor cells are of a different genotype than the non-tumor cells. One important event for tumor initiation is LOH of tumor suppressor genes, during which the wild type copy of a particular gene is disrupted in an already heterozygous subject, resulting in a few homozygous mutant cells.

Mouse models have been used to provide mechanistic insights of tumorigenesis, however a mouse model that is genetically mosaic can provide a unique opportunity to study tumorigenesis in particular tissues and during different developmental stages, in comparison with a neighboring wild type cell. The MADM system (see U.S. Pat. No. 7,282,621; Zong et al., Cell, 121:479-492, 2005; Muzumdar et al., Proc. Natl. Acad. Sci., 104:4495-4500, 2007; all of which are incorporated herein by reference) is a system that allows the generation of site-directed chromosome recombination in somatic cells, thereby generating sporadic mutant cells in an otherwise normal mouse (mosaic) and mimicking the sporadic LOH of tumor suppressor genes in human cancer physiology.

The MADM system has the ability to differentially label both the homozygous mutant cells and sibling wild type cells in the genetically mosaic animal. The MADM system labels one or more particular cells in a subject in order to investigate the detailed morphology and function of the cells. As one or more specific genes in the labeled cells can be modified, for example by knock-in, knock-out, or targeted mutation events, changes to morphology and/or function of the labeled cells can be observed in the cell as a result of the genetic modification.

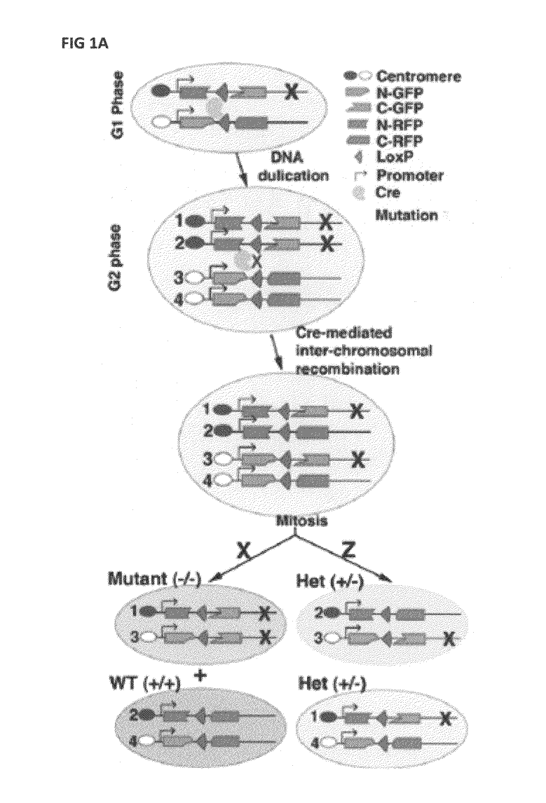

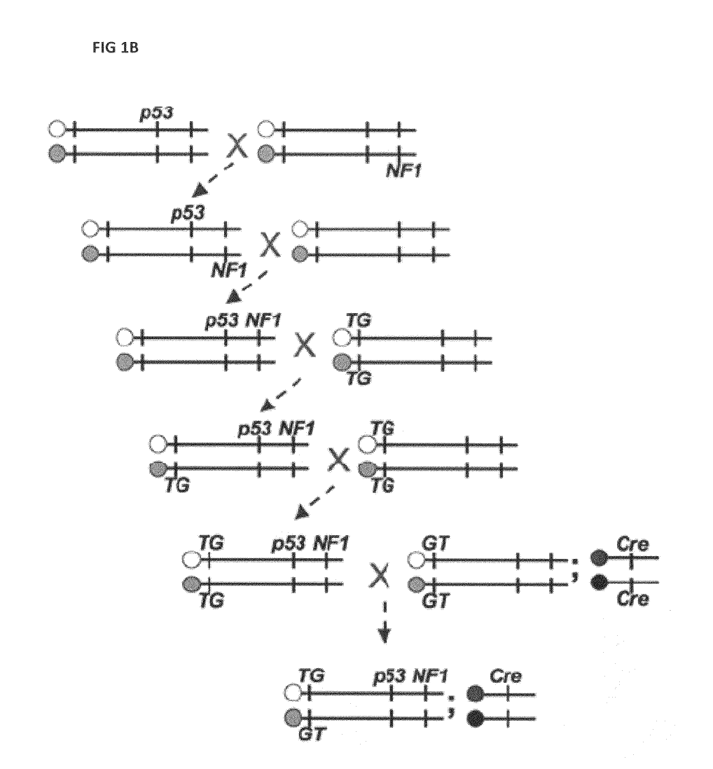

MADM utilizes Cre recombinase-dependent interchromosomal recombination at loxP sites at homologous locations on a chromosome pair to generate a limited number of differentially labeled homozygous mutant and wild type cells in a heterozygous background. FIG. 1A illustrates the MADM system. Two reciprocally chimeric marker genes are knocked-in by homologous recombination at an identical locus on homologous chromosomes. “Reciprocally chimeric,” indicates that one chimeric marker gene includes a portion of a marker gene (such as an N-terminal portion) and the other (“reciprocal”) chimeric marker gene includes the remainder of the marker gene (such as a C-terminal portion), such that when recombination occurs between two reciprocally chimeric marker genes a functional marker gene is generated. By way of example, a chimeric marker gene could encode amino acids 1-50 of a 100 amino acid marker protein and the reciprocal chimeric marker gene could encode amino acids 51-100 of the same marker protein, such that upon recombination, a gene encoding amino acids 1-100 of the marker protein is generated. In other examples, a chimeric marker gene could encode a proportion of the marker protein (such as one-half, one-third, one-quarter, one-tenth, and so on) and the reciprocal chimeric marker gene could encode the remaining proportion of the marker protein (such as the remaining one-half, two-thirds, three-quarters, nine-tenths, and so on).

In particular examples, the chimeric marker genes are reciprocally chimeric and include an N-terminal portion of one marker gene (for example a GFP, such as EGFP) and a C-terminal portion of a second marker gene (for example, a RFP, such as tdT). The N- and C-terminal portions of each of the reciprocally chimeric marker genes are separated by an intron that includes one loxP site. In some examples, in the absence of Cre-mediated recombination, the chimeric genes do not encode a functional protein, because the coding sequences are interrupted by the intron in different reading frames. However, when Cre-mediated recombination occurs, two functional marker genes are generated.

In some examples, Cre recombinase is introduced to the genome of a mouse including a knocked-in marker gene by crossing with a mouse line that is transgenic for Cre under the control of a heterologous promoter. The promoter may be a constitutively expressed promoter, a temporally limited promoter, a ubiquitously expressed promoter, a tissue- or cell type-specific promoter, an inducible promoter, or a combination of two or more promoter types. In some examples, the Cre recombinase is operably linked to a promoter that is active in one or more specific tissue or cell type of interest (such as skin, melanocytes, mammary tissue, prostate, neurons, neural precursors, astrocytes, and so on). In a particular example, the Cre recombinase is operably linked to a promoter that is active in neural stem cells and/or glial precursor cells (such as a GFAP promoter, a Nestin promoter, or a NG2 promoter).

In other examples, the Cre recombinase may be operably linked to an inducible promoter, such as a tetracycline inducible promoter. If a tetracycline inducible promoter is utilized, Cre recombinase is operably linked to a promoter that includes a tetracycline response element (TRE) and the genome of the transgenic mouse also includes a transgene that expresses a tetracycline controlled transactivator under the control of a heterologous promoter (such as a tissue- or cell type-specific promoter). Cre recombinase expression can be induced by administering tetracycline (or a tetracycline analog, such as doxycycline) to the transgenic mouse (for example, at a particular time point of interest).

The Cre recombinase, when expressed, induces recombination between the loxP sites in the reciprocally chimeric marker genes, producing functional marker genes (for example, functional GFP and RFP). If this recombination occurs after DNA replication (during G2 phase), two types of chromosome segregation are possible during the subsequent mitosis (FIG. 1A). If X segregation occurs (two recombinant sister chromatids segregate into different daughter cells), one of the resulting daughter cells will be capable of expressing one of the markers (for example, GFP) and the other daughter cell will be capable of expressing the other marker (for example, RFP). If Z segregation occurs (two recombinant sister chromatids segregate into the same daughter cell), one of the resulting daughter cells will be capable of expressing both markers (for example, both GFP and RFP) while the other daughter cell will contain both chimeric marker genes (like the parent cell).

If one of the chromosomes with the knocked-in chimeric marker gene has one or more mutations of interest (such as one or more gene knockout, gene knock-in, insertion, deletion, or other mutation) distal to the knock-in site, G2 recombination followed by X segregation will generate a marked (labeled) daughter cell homozygous for the mutation(s), and a differentially marked (labeled) daughter cell that is wild type, thereby creating a labeled genetic mosaic (FIG. 1A). All progeny of these daughter cells will also contain the functional marker gene, which allows the developmental lineage and fate of these cells to be followed. For example, if the marker genes are GFP and RFP, and a knockout allele is introduced distally on the same chromosome that includes the N-RFP_loxP_C-GFP construct, G2 recombination and X segregation will produce a green (GFP) cell homozygous for the knockout and a red (RFP) cell homozygous wild type.

In a particular example of the MADM system described herein, one or more knockout allele is introduced into the mouse genome as a “floxed” allele, such that the null mutation does not arise unless Cre recombinase is expressed. A “floxed” allele is a gene that includes at least two loxP sites present in the same orientation, such that the sequence between the loxP sites is excised by Cre recombinase. In particular examples, the loxP sites are positioned in the gene so that upon excision of the intervening sequence by Cre recombinase, a non-functional or “knockout” allele is created. Floxed alleles allow the creation of a conditional mutation (such as a gene knockout) because the timing or location of Cre recombinase expression can be controlled (for example by expressing Cre from a tissue- and/or developmental stage-specific promoter or an inducible promoter). In some examples, use of a floxed allele to create a conditional gene knockout is useful because mutation of a constitutive or ubiquitously expressed gene can result in lethality or other undesirable phenotypes. Creation of such conditional gene knockouts utilizing the Cre-loxP system is well known in the art. See, e.g., Orban et al., Proc. Natl. Acad. Sci. USA 89:6861-6865, 1992; Transgenic Mouse Methods and Protocols, Eds. Hofker and van Duersen, Humana Press, 2002; Manipulating the Mouse Embryo, Nagy et al., Cold Spring Harbor Laboratory Press, Third Edition, 2002. The placement of the loxP sites may be designed in order to delete the entire gene of interest or a portion of the gene of interest (for example, one or more exons) to produce a non-functional gene.

In some examples, the MADM mice described herein include a floxed NF1 gene. In the absence of expression of Cre recombinase (for example, in a mouse lacking a Cre transgene, or in a cell that does not express Cre recombinase) the floxed NF1 gene is functional and is equivalent to a wild type NF1 gene. In a particular example, the floxed NF1 gene includes loxP sites flanking exons 31 and 32 of the NF1 gene (see, e.g., Zhu et al., Genes Dev. 15:859-876, 2001). In the presence of expression of Cre recombinase (such as in a mouse including a Cre transgene or in a cell that expresses Cre recombinase), the portion of the NF1 gene between the loxP sites is deleted and a non-functional (“knockout”) NF1 gene is generated. In particular examples, the floxed NF1 gene is introduced in the genome of a mouse that includes the MADM system (for example, a chromosome 11 MADM system).

B. Mouse Tumor Model

Disclosed herein are mouse tumor models which utilize the MADM system on mouse chromosome 11 to generate a sporadic knockout of one or more tumor suppressor (such as p53, NF1, or both p53 and NF1), while simultaneously labeling the homozygous null cells and the sibling wild type cells. In one example, the resulting transgenic mice develop glioma (for example, grade III astrocytoma or grade IV glioblastoma multiforme) and exhibit diffuse mutant cell hyperplasia prior to tumor development.

In one embodiment, the genome of a MADM transgenic mouse includes a first nucleic acid molecule which includes a first promoter operably linked to a nucleic acid molecule encoding an N-terminal portion of a green fluorescent protein (GFP) and a C-terminal portion of a tdTomato (tdT) fluorescent protein, wherein the N-terminal portion of the GFP and the C-terminal portion of the tdT protein are separated by a β-globin intron including a first loxP site (N-GFP_loxP_C-tdT), and wherein the first nucleic acid molecule is present at a locus of a first chromosome 11 of a chromosome 11 pair, and a second nucleic acid molecule which includes a second promoter operably linked to a nucleic acid molecule encoding an N-terminal portion of the tdT protein and a C-terminal portion of the GFP, wherein the N-terminal portion of the tdT protein and the C-terminal portion of the GFP are separated by the β-globin intron including a second loxP site (N-tdT_loxP_C-GFP), and wherein the second nucleic acid molecule is present at a homologous locus of a second chromosome 11 of the chromosome 11 pair.

In some embodiments, the genome of the MADM transgenic mouse includes a heterozygous null mutation in the p53 gene and a heterozygous floxed NF1 gene, both of which were originally introduced in the mouse genome by homologous recombination and introduced on the MADM-bearing chromosome by standard breeding techniques. The “foxed” NF1 gene is an NF1 gene that includes a pair of loxP sites in the same orientation, such that in the presence of Cre recombinase the sequence between the loxP sites will be excised, resulting in the formation of an NF1 null allele. In the absence of Cre recombinase expression, the floxed NF1 gene functions as a wild type NF1 allele. In a particular example, the floxed NF1 gene is that present in the genome of transgenic mouse of Stock No. 01XM4 (NC1 Mouse Repository, Frederick, Md.). See, e.g., Zhu et al., Genes Dev. 15:859-876, 2001. In other embodiments, the genome of the MADM transgenic mouse includes a heterozygous null mutation in the p53 gene (in the absence of a NF1 mutation) which was originally introduced in the mouse genome by homologous recombination and introduced on the MADM-bearing chromosome by standard breeding techniques. In a still further embodiment, the genome of the MADM transgenic mouse includes a heterozygous foxed NF1 gene (in the absence of a p53 mutation), which was originally introduced in the mouse genome by homologous recombination and introduced on the MADM-bearing chromosome by standard breeding techniques.

In some examples, the genome of the transgenic mouse further includes a nucleic acid molecule encoding a Cre recombinase operably linked to a promoter (such as a promoter that is active in neural stem cells and/or glial precursor cells, for example, a GFAP, Nestin, or NG2 promoter). In some examples, the Cre recombinase is introduced into the genome of the MADM mouse by crossing a transgenic mouse including a N-GFP_loxP_C-RFP construct (GT mouse) with a mouse including Cre recombinase operably linked to a tissue-specific, inducible, and/or temporally specific promoter. In other examples, the Cre recombinase is introduced into the genome of the MADM mouse by crossing a transgenic mouse including a N-RFP_loxP_C-GFP construct (TG mouse) with a mouse including Cre recombinase operably linked to a tissue-specific, inducible, and/or temporally specific promoter. In further examples, the Cre recombinase is introduced utilizing extra-chromosomal DNA, for example, a viral vector delivery system, which in some instances may subsequently integrate into the mouse genome. Viral vectors are well known in the art, and in particular examples, include avian leukosis virus (such as Rous sarcoma virus), adenovirus, lentivirus, or adeno-associated virus vectors.

In particular examples, the promoter operably linked to the Cre recombinase is selected to direct tissue- or cell type-specific expression. Exemplary promoters are provided in Table 1. Mice that are transgenic for Cre recombinase operably linked to a promoter can be generated one of skill in the art by conventional techniques. Such mice are also commercially available, for example from The Jackson Laboratory (Bar Harbor, Me.), Taconic Farms (Hudson, N.Y.), and the National Cancer Institute Mouse Repository (Frederick, Md.). A database of Cre transgenic mice is also available on the World Wide Web at nagy.mshri.on.ca/cre_new/index.php.

TABLE 1
Exemplary promoters and cell or tissue types for
tissue-specific expression of Cre recombinase

| Promoter | Tissue or cell type |
|---|---|
| GFAP | Neural stem cells, astrocytes |
| Nestin | Neural stem cells |
| NG2 | Oligodendrocyte precursors |
| Brain lipid binding protein | Neural stem cells |
| (BLBP; FABP7) | |
| Keratin 14 | Skin |
| Tyrosinase | Melanocytes |
| Micropthalmia-associated | Melanocytes |
| transcription factor (Mitf) | |
| Probasin (PB) | Prostate |
| Pancreatic and duodenal homeobox 1 | Pancrease |
| (Pdx1) | |
| Fatty acid binding protein 1 (Fabp1) | Intestinal epithelium |
| Whey acidic protein | Mammary tissue |
| Mouse mammary tumor virus | Mammary tissue |

Cre recombinase-promoted somatic recombination at loxP sites occurs in at least one cell of the transgenic mouse including a nucleic acid molecule encoding a Cre recombinase operably linked to a promoter. In one example, a heterozygous p53 null mutation and a heterozygous floxed NF1 gene are present on the second chromosome 11 of the chromosome 11 pair (for example, in cis with the N-tdT_loxP_C-GFP knock-in) and following at least one recombination event, the transgenic mouse includes at least a first cell including a first recombined nucleic acid molecule encoding a functional GFP, a homozygous null mutation in the p53 gene, and a homozygous null mutation in the NF1 gene and at least a second cell including a second recombined nucleic acid molecule encoding a functional tdT protein, a homozygous wild type p53 gene, and a homozygous wild type NF1 gene. In another example, a heterozygous p53 null mutation and a floxed NF1 gene are present on the first chromosome of the chromosome 11 pair (for example, in cis with the N-GFP_loxP_C-tdT knock-in) and following at least one recombination event, the transgenic mouse includes at least a first cell including a first recombined nucleic acid molecule encoding a functional tdT, a homozygous null mutation in the p53 gene, and a homozygous null mutation in the NF1 gene and a least a second cell including a second recombined nucleic acid molecule encoding a functional GFP protein, a homozygous wild type p53 gene, and a homozygous wild type NF1 gene.

In another example, a heterozygous p53 null mutation (in the absence of a NF1 mutation) is present on the second chromosome 11 of the chromosome 11 pair (for example, in cis with the N-tdT_loxP_C-GFP knock-in) and following at least one recombination event, the transgenic mouse includes at least a first cell including a first recombined nucleic acid molecule encoding a functional GFP and a homozygous null mutation in the p53 gene and at least a second cell including a second recombined nucleic acid molecule encoding a functional tdT protein and a homozygous wild type p53 gene. In another example, a heterozygous p53 null mutation (in the absence of a NF1 mutation) is present on the first chromosome of the chromosome 11 pair (for example, in cis with the N-GFP_loxP_C-tdT knock-in) and following at least one recombination event, the transgenic mouse includes at least a first cell including a first recombined nucleic acid molecule encoding a functional tdT and a homozygous null mutation in the p53 gene and a least a second cell including a second recombined nucleic acid molecule encoding a functional GFP protein and a homozygous wild type p53 gene.

In a further example, a heterozygous floxed NF1 gene (in the absence of a p53 mutation) is present on the second chromosome 11 of the chromosome 11 pair (for example, in cis with the N-tdT_loxP_C-GFP knock-in) and following at least one recombination event, the transgenic mouse includes at least a first cell including a first recombined nucleic acid molecule encoding a functional GFP and a homozygous null mutation in the NF I gene and at least a second cell including a second recombined nucleic acid molecule encoding a functional tdT protein and a homozygous wild type NF1 gene. In another example, a heterozygous floxed NF1 gene (in the absence of a p53 mutation) is present on the first chromosome of the chromosome 11 pair (for example, in cis with the N-GFP_loxP_C-tdT knock-in) and following at least one recombination event, the transgenic mouse includes at least a first cell including a first recombined nucleic acid molecule encoding a functional tdT and a homozygous null mutation in the NF1 gene and a least a second cell including a second recombined nucleic acid molecule encoding a functional GFP protein and a homozygous wild type NF1 gene.

The disclosed transgenic mice are generated by standard methods of homologous recombination and transgene introduction, as well as standard mouse breeding techniques (see e.g., Nagy et al., Manipulating the Mouse Embryo, 3^{rd} edition, Cold Spring Harbor Press, 2002). The chromosome 11 MADM mice are produced by first creating chimeric N-GFP_loxP_C-RFP and N-RFP_loxP_C-GFP gene targeting constructs. The N-GFP_loxP_C-RFP construct is generated by joining a promoter (such as a constitutively active promoter, for example, a CMV β-actin enhancer-promoter), a nucleic acid encoding an N-terminal portion of a GFP (for example, enhanced GFP (EGFP)), an intron (for example, a β-globin intron) containing one loxP site, a nucleic acid encoding a C-terminal portion of a RFP (for example, tdT), and a poly(A) signal (for example, a SV40 T antigen poly(A) signal). The N-RFP_loxP_C-GFP construct is generated by joining a promoter (such as a constitutively active promoter, for example, a CMV β-actin enhancer-promoter), a nucleic acid encoding an N-terminal portion of a RFP (for example, tdT), an intron (for example, a β-globin intron) containing one loxP site, a nucleic acid encoding a C-terminal portion of a GFP (for example, EGFP), and a poly(A) signal (for example, a SV40 T antigen poly(A) signal).

Genes for fluorescent proteins, such as GFPs and RFPs are well known in the art. See, e.g., Prasher et al., Gene 111:229-233, 1992; Shaner et al., Nature Biotechnol. 22:1567-1572, 2004; U.S. Pat. No. 6,172,188. Nucleic acids encoding fluorescent proteins are also commercially available, for example from Clontech (Mountain View, Calif.) or Invitrogen (Carlsbad, Calif.). In a particular example, the GFP includes EGFP (for example, mut4-EGFP; Okada et al., Exp. Neurol. 156:394-406, 1999). GFP sequences, such as EGFP sequences are publicly available. Exemplary EGFP nucleic acid and amino acid sequences include GenBank Accession Nos. U57606 (nucleotides 613-1410) and AAB08058, respectively (incorporated herein by reference as present in GenBank on Aug. 10, 2009). In one example, mut4-EGFP is a modified EGFP which includes the mutations V163A and S175G. In another specific example, the RFP includes tdTomato (Shaner et al., Nature Biotechnol. 22:1567-1572, 2004). RFP (for example tdT) sequences are publicly available. In one example, tdT nucleic acid and amino acid sequences include GenBank Accession Nos. AY678269 and AAV52169, respectively (incorporated herein by reference as present in GenBank on Aug. 10, 2009). One of skill in the art can select appropriate fluorescent proteins for the transgenic mice and methods described herein.

The N- and C-terminal portions of each construct are chosen such that if Cre-mediated recombination occurs between the N-GFP_loxP_C-RFP construct and the N-RFP_loxP_C-GFP construct a functional GFP and a functional RFP results. In one example, the nucleic acid molecules encoding N- and C-terminal portions of the GFP and RFP are interrupted by the intron in different reading frames in order to prevent the production of functional chimeric GFP-RFP proteins. In some examples, the location of the split between the N- and C-terminal portions of the GFP and/or RFP is chosen in order to optimize splicing of the intervening intron, for example, between two guanine nucleotides in the coding sequence. In addition, if the N- and C-terminal portions are interrupted by the intron in different reading frames, the split may be placed closer to the N-terminal of the protein (for example, in the N-terminal half or in the N-terminal quarter of the protein) to reduce potential production of non-functional (“junk”) chimeras if splicing of the protein occurs in the absence of Cre-mediated recombination. Placing the split closer to the N-terminal will introduce an early stop codon if inappropriate splicing occurs and reduce the size of any non-functional protein that is produced.

In some examples, N-GFP_loxP_C-tdT and N-tdT_loxP_C-GFP constructs are produced. In a particular example, the GFP (such as EGFP, for example, mut4-EGFP) is split such that the N-GFP portion includes amino acids 1-95 and the C-GFP portion includes amino acids 96-239. In some examples, the split is introduced in reading frame 3. In another particular example, the RFP (such as tdT) is split such that the N-terminal tdT portion includes the first amino acid and the C-terminal tdT portion includes the remainder of the tdT protein (amino acids 2-476). In some examples, the split is introduced such that the reading frame is shifted +1 (for example, the N-terminal tdT portion is encoded by the nucleotides ATGG).

In one example, the N-GFP_loxP_C-tdT and N-tdT_loxP_C-GFP constructs are each inserted into a chromosome 11 targeting vector. In a particular example, the targeting vector directs insertion of the N-GFP_loxP_C-tdT and N-tdT_loxP_C-GFP constructs at nucleotides 145273-145274 of mouse chromosome 11 (mouse genome build 37, GenBank Accession No. Mm11_{—}39555_{—}37; incorporated herein by reference as present in GenBank on Aug. 10, 2009). Two transgenic mouse lines are created by standard homologous recombination in mouse embryonic stem (ES) cells; one line carries a knock-in of the N-GFP_loxP_C-tdT construct (a “GT” mouse), and the other line carries a knock-in of the N-tdT_loxP_C-GFP construct (a “TG” mouse).

Several criteria are considered in choosing the chromosomal locus to knock-in the chimeric marker genes for the MADM system (the “MADM locus”). In some examples, the MADM locus is relatively close to the centromere (such as about 1-5 cM, for example about 3-4 cM from the centromere). This allows the study of most of the genes on the chromosome of interest using the MADM system (for example, TSGs on mouse chromosome 11, such as NF1, p53, NF2, Hic1, Mnt, Lgl1, Lgl2, and BRCA1). However, the MADM locus may be anywhere on the chromosome, as long as the gene of interest is located between the locus and the telomere. Also, the MADM locus should not disrupt any coding sequences, for example, the locus is between genes (in an intergenic region). Intergenic regions may be identified using available mouse genome maps (for example, the Ensembl mouse genome browser; available on the World Wide Web at ensembl.org/Mus_musculus/Info/Index). The MADM locus also should not disrupt any non-coding regions that are conserved between species (such as regions conserved between mouse, rat, and human), as these may include important regulatory sequences. Regions of conserved sequence between species may be identified by comparing the region of interest from different species using available software applications, such as VISTA (Frazer et al., Nucl. Acids Res. 32:W273-279, 2004; available on the World Wide Web at genome.lbl.gov/vista/mvista/submit.shtml). The MADM locus should also be in an area of open chromatin structure, as this is believed to allow both access of Cre recombinase for efficient inter-chromosomal recombination and expression of the marker genes after recombination. In some examples, the MADM locus is near one or more housekeeping genes (for example, an intergenic region between two housekeeping genes). Also, the MADM locus should not include repetitive DNA elements (for example, SINE elements, LINE-1 elements, microsatellite repeats, or minisatellite repeats). Repetitive DNA elements can be identified in a sequence of interest using available software applications, for example, RepeatMasker (Smit et al., unpublished data, Current Version: open-3.2.8 (RMLib:20090604); available on the World Wide Web at repeatmasker.org/cgi-bin/WEBRepeatMasker) or CENSOR (Kohany et al., BMC Bioinform. 7:474, 2006; available on the World Wide Web at girinst.org/censor/). Finally, the MADM locus should have a balanced GCAT composition (such as approximately 25% of each nucleotide in the locus), to avoid potential secondary structure that may interfere with gene targeting and should not include long stretches of any single nucleotide.

In some examples, the chromosomal locus for the knock-in of the MADM chimeric N-GFP_loxP_C-tdT nucleic acid molecule and the chimeric N-tdT_loxP_C-GFP nucleic acid molecule is about 3-4 cM from the centromere of mouse chromosome 11. In a particular example, the MADM chimeras are knocked-in to chromosome 11 in the intergenic region between the Eif4enif1 gene and the Drg1 gene. In one example, the Eif4enif1 gene is located on mouse chromosome 11 at nucleotides 102765-144584 (see e.g., GenBank Accession No. NT_{—}039515; incorporated herein by reference as present in GenBank on Aug. 10, 2009) and the Drg1 gene is located on mouse chromosome 11 at nucleotides 149925-166389 (complement; see e.g., GenBank Accession No. NT 039515; incorporated herein by reference as present in GenBank on Aug. 10, 2009).

Mouse strains are named using the following nomenclature system. A “,” is used to indicate two genes on the same chromosome (in cis). A “/” is used to designate genes on separate homologous chromosomes (in trans). Finally, a “;” is used to separate genes that are not linked to the preceding chromosome.

In one example, a heterozygous null mutation for the p53 gene and a foxed NF1 gene are introduced by crossing one of the transgenic mice described above (such as a GT mouse or a TG mouse) with a mouse that is heterozygous for both the p53 null mutation and the foxed NF1 gene on the same chromosome 11 (in cis) to obtain trans-heterozygous MADM/p53KO, NF1FLOX mice. This mouse is crossed to WT mice to generate MADM, p53KO, NF1FLOX mice, in which germline homologous recombination results in the cis configuration of these genetic components. FIG. 1B shows an exemplary breeding scheme to generate these mice. The mouse that is heterozygous null for p53KO and NF1FLOX may be generated by standard breeding methods, such as crossing a mouse that is heterozygous for a p53 knockout allele (such as Stock No. 002101, The Jackson Laboratory, Bar Harbor, Me.) and a mouse that is heterozygous for a floxed NF1 knockout allele (such as Stock No. 01XM4, NCI Mouse Repository, Frederick, Md.).

In another example, a heterozygous null p53 mutation is introduced by crossing one of the transgenic mice described above (such as a GT mouse or a TG mouse) with a mouse that is heterozygous for a p53 null mutation (in the absence of a NF1 mutation) to obtain trans-heterozygous MADM/p53KO mice. This mouse is crossed to WT mice to generate MADM, p53KO mice, in which germline homologous recombination results in the cis configuration of these genetic components. In another example, a heterozygous floxed NF1 gene is introduced by crossing one of the transgenic mice described above (such as a GT mouse or a TG mouse) with a mouse that is heterozygous for a floxed NF1 gene (in the absence of a p53 mutation) to obtain trans-heterozygous MADM/NF1FLOX mice. This mouse is crossed to WT mice to generate MADM, NF1FLOX mice, in which germline homologous recombination results in the cis configuration of these genetic components.

In another particular example, a transgenic mouse with an N-tdT_loxP_C-GFP knock-in on chromosome 11 (TG mouse) is crossed with a transgenic mouse that is heterozygous for a p53 null mutation and a floxed NF1 gene in cis. Progeny from the cross which have the TG knock-in and the p53 null mutation and floxed NF1 alleles on chromosome 11 in cis are selected. These mice are designated TG, p53KO, NF1FLOX mice.

In a further particular example, a transgenic mouse with an N-tdT_loxP_C-GFP knock-in on chromosome 11 (TG mouse) is crossed with a transgenic mouse that is heterozygous for a p53 null mutation (in the absence of a NF1 mutation). Progeny from the cross which have the TG knock-in and the p53 null mutation on chromosome 11 in cis are selected. These mice are designated TG, p53KO mice. In a further particular example, a transgenic mouse with an N-tdT_loxP_C-GFP knock-in on chromosome 11 (TG mouse) is crossed with a transgenic mouse that is heterozygous for a floxed NF1 gene (in the absence of a p53 mutation). Progeny from the cross which have the TG knock-in and the floxed NF1 gene on chromosome 11 in cis are selected. These mice are designated TG, NF1FLOX mice.

In some examples, a transgenic mouse that includes the N-GFP_loxP_C-tdT construct (GT) and a Cre recombinase (such as a Cre recombinase operably linked to a tissue-specific, temporally-specific, and/or inducible promoter) is separately generated by crossing a GT transgenic mouse with a mouse transgenic for Cre recombinase (such as Cre recombinase that is expressed under the control of a promoter that is active in glial cells, for example, a GFAP promoter). See, e.g., Zhuo et al., Genesis 31:85-94, 2001. These mice are designated GT; Cre mice. The Cre recombinase transgene may be on any chromosome. A p53-NF1 MADM transgenic mouse is finally generated by crossing the TG, p53KO, NF1FLOX and GT; Cre mice to produce TG, p53KO, NF1FLOX/GT; Cre mice. A p53 MADM transgenic mouse is finally generated by crossing the TG, p53KO and GT; Cre mice to produce TG, p53KO/GT; Cre mice. An NF1 MADM transgenic mouse is finally generated by crossing the TG, NF1FLOX and GT; Cre mice to produce TG, NF1FLOX/GT; Cre mice.

In some examples, the TG, p53KO, NF1FLOX/GT; Cre mice undergo somatic recombination at the loxP sites located in the TG and GT transgenes in one or more of the cells expressing the Cre recombinase transgene (for example, in neural stem cells and/or glial precursor cells). The TG, p53KO, NF1FLOX/GT; Cre mice also undergo recombination at the loxP sites in the floxed NF1 gene in one or more of the cells expressing the Cre recombinase transgene, resulting in excision of a portion of the NF1 gene and production of an NF1 knockout mutation. In particular examples, a cell in which somatic recombination occurs goes through X segregation. In this situation, one of the resulting daughter cells includes a recombined functional GFP and is homozygous for both the p53 and NF1 knockout mutations; this cell (and all of its progeny) is detectable as a green cell under appropriate conditions. In this situation, the other resulting daughter cell includes a recombined functional tdT protein and is homozygous wild type for p53 and NF1; this cell (and all of its progeny) is detectable as a red cell under appropriate conditions.

In other examples, the TG, p53KO/GT; Cre mice undergo somatic recombination at the loxP sites located in the TG and GT transgenes in one or more of the cells expressing the Cre recombinase transgene (for example, in neural stem cells and/or glial precursor cells). In particular examples, a cell in which somatic recombination occurs goes through X segregation. In this situation, one of the resulting daughter cells includes a recombined functional GFP and is homozygous for the p53 knockout mutations; this cell (and all of its progeny) is detectable as a green cell under appropriate conditions. In this situation, the other resulting daughter cell includes a recombined functional tdT protein and is homozygous wild type for p53; this cell (and all of its progeny) is detectable as a red cell under appropriate conditions.

In further examples, the TG, NF1FLOX/GT; Cre mice undergo somatic recombination at the loxP sites located in the TG and GT transgenes in one or more of the cells expressing the Cre recombinase transgene (for example, in neural stem cells and/or glial precursor cells). The TG, NF1FLOX/GT; Cre mice also undergo recombination at the loxP sites in the floxed NF1 gene in one or more of the cells expressing the Cre recombinase transgene, resulting in excision of a portion of the NF1 gene and production of an NF1 knockout mutation. In particular examples, a cell in which somatic recombination occurs goes through X segregation. In this situation, one of the resulting daughter cells includes a recombined functional GFP and is homozygous for the NF1 knockout mutation; this cell (and all of its progeny) is detectable as a green cell under appropriate conditions. In this situation, the other resulting daughter cell includes a recombined functional tdT protein and is homozygous wild type for NF1; this cell (and all of its progeny) is detectable as a red cell under appropriate conditions.

In another example, a transgenic mouse with an N-GFP_loxP_C-tdT knock-in on chromosome 11 (GT mouse) is crossed with a transgenic mouse that is heterozygous for a p53 null mutation and a floxed NF1 gene in cis. Progeny from the cross which have the GT knock-in and the p53 null mutation and floxed NF1 gene on chromosome 11 in cis are selected. These mice are designated GT, p53KO, NF1FLOX mice. In a further example, a transgenic mouse with an N-GFP_loxP_C-tdT knock-in on chromosome 11 (GT mouse) is crossed with a transgenic mouse that is heterozygous for p53 null mutation. Progeny from the cross which have the GT knock-in and the p53 null mutation on chromosome 11 in cis are selected. These mice are designated GT, p53KO mice. In a further example, a transgenic mouse with an N-GFP_loxP_C-tdT knock-in on chromosome 11 (GT mouse) is crossed with a transgenic mouse that is heterozygous for a floxed NF1 gene. Progeny from the cross which have the GT knock-in and the floxed NF1 gene on chromosome 11 in cis are selected. These mice are designated GT, NF1FLOX mice.

A transgenic mouse that includes the N-tdT_loxP_C-GFP construct (TG) and a Cre recombinase (such as a Cre recombinase operably linked to a tissue-specific, temporally-specific, and/or inducible promoter) is separately generated by crossing a TG transgenic mouse with a mouse transgenic for Cre recombinase (such as Cre recombinase that is expressed under the control of a promoter that is active in glial cells, for example, a GFAP promoter). See, e.g., Zhuo et al., Genesis 31:85-94, 2001. These mice are designated TG; Cre mice. The Cre recombinase transgene may be on any chromosome. A p53-NF1 MADM transgenic mouse is finally generated by crossing the GT, p53KO, NF1FLOX and TG; Cre mice to produce GT, p53KO, NF1FLOX/TG; Cre mice. A p53 MADM transgenic mouse is finally generated by crossing the GT, p53KO and TG; Cre mice to produce GT, p53KO/TG; Cre mice. An NF1 MADM transgenic mouse is finally generated by crossing the GT, NF1FLOX and TG; Cre mice to produce GT, NF1 FLOX/TG; Cre mice.

In some examples, the GT, p53KO, NF1FLOX/TG; Cre mice undergo somatic recombination at the loxP sites located in the GT and TG transgenes in one or more of the cells expressing the Cre recombinase transgene (for example, in neural stem cells and/or glial precursor cells). The GT, p53KO, NF1FLOX/TG; Cre mice also undergo recombination at the loxP sites in the floxed NF1 gene in one or more of the cells expressing the Cre recombinase transgene, resulting in excision of a portion of the NF1 gene and production of an NF1 knockout mutation. In particular examples, a cell in which somatic recombination occurs goes through X segregation. In this situation, one of the resulting daughter cells includes a recombined functional tdT and is homozygous for both the p53 and NF1 knockout mutations; this cell (and all of its progeny) is detectable as a red cell under appropriate conditions. In this situation, the other resulting daughter cell includes a recombined functional GFP and is homozygous wild type for p53 and NF1; this cell (and all of its progeny) is detectable as a green cell under appropriate conditions.

In other examples, the GT, p53KO/TG; Cre mice undergo somatic recombination at the loxP sites located in the GT and TG transgenes in one or more of the cells expressing the Cre recombinase transgene (for example, in neural stem cells and/or glial precursor cells). In particular examples, a cell in which somatic recombination occurs goes through X segregation. In this situation, one of the resulting daughter cells includes a recombined functional tdT and is homozygous for the p53 knockout mutation; this cell (and all of its progeny) is detectable as a red cell under appropriate conditions. In this situation, the other resulting daughter cell includes a recombined functional GFP and is homozygous wild type for p53; this cell (and all of its progeny) is detectable as a green cell under appropriate conditions.

In some examples, the GT, NF1FLOX/TG; Cre mice undergo somatic recombination at the loxP sites located in the GT and TG transgenes in one or more of the cells expressing the Cre recombinase transgene (for example, in neural stem cells and/or glial precursor cells). The GT, NF1FLOX/TG; Cre mice also undergo recombination at the loxP sites in the floxed NF1 gene in one or more of the cells expressing the Cre recombinase transgene, resulting in excision of a portion of the NF1 gene and production of an NF1 knockout mutation. In particular examples, a cell in which somatic recombination occurs goes through X segregation. In this situation, one of the resulting daughter cells includes a recombined functional tdT and is homozygous for the NF1 knockout mutation; this cell (and all of its progeny) is detectable as a red cell under appropriate conditions. In this situation, the other resulting daughter cell includes a recombined functional GFP and is homozygous wild type for NF1; this cell (and all of its progeny) is detectable as a green cell under appropriate conditions.

In some examples, the transgenic mice disclosed herein develop one or more tumors. Due to the nature of the MADM system, as discussed above, gene knockout (such as p53, NF1, or both p53 and NF1 homozygous null) arises only in cells that express Cre recombinase. Therefore, tumor formation depends, at least in part on the spatio-temporal expression of Cre recombinase in the transgenic mouse. One of skill in the art can select a Cre recombinase construct with an appropriate promoter to drive expression in a selected tissue, cell type, and/or developmental stage (discussed above) where tumor formation is desired.

In particular examples, a glioma develops from one or more cells that have undergone somatic recombination and are homozygous null for p53 and NF1 and express Cre recombinase from a hGFAP, nestin, or NG2 promoter. In one example, tumor pathology (for example, pseudopalisading necrosis, peri-neuronal satellitosis, hyperplastic vasculature, and presence of markers of cell division) is detectable in the disclosed transgenic mice at around 4-5 months of age. In additional examples, the disclosed transgenic mice exhibit a diffuse overproliferation of cells that are homozygous null for p53 and NF1 as compared with cells that are wild type for p53 and NF1 (for example, about 5-fold, 10-fold, 20-fold, 50-fold, 100-fold, or 200-fold more homozygous null cells compared to wild type cells). In some examples, the over-proliferation of p53 and NF1 null cells is present before the development of tumor pathology (for example, at about 10 days, 21 days, one month, two months, or three months post-natal).

In other examples, a melanoma develops from one or more cells that have undergone somatic recombination and are homozygous null for p53 and NF1 and express Cre recombinase from a melanocyte-specific promoter. In further examples, a neurofibroma develops from one or more cells that have undergone somatic recombination and are homozygous null for NF1 and express Cre recombinase from a Schwann cell-specific promoter. One of skill in the art can readily identify other tumors that may arise in the disclosed MADM mice in combination with particular expression of Cre recombinase.

### IV. Methods for Identifying Compounds for Treating or Preventing Tumors

Disclosed herein are methods for identifying compounds for treating or preventing tumors (such as glioma) utilizing the transgenic mice described in Section III, above. These methods include cell-based assays and in vivo assays, separately or in combination.

In some examples, the methods include culturing at least one cell including a nucleic acid molecule encoding a functional GFP (or tdT), a homozygous null p53 mutation, and a homozygous null NF1 mutation, which is isolated or purified from a p53-NF1 MADM transgenic mouse as described herein, contacting the cell with at least one test compound, determining the phenotype of the cell (or a population of cells), and selecting a compound that alters (for example, increases or decreases a measurable characteristic) the phenotype as compared to a control, thereby identifying a compound for treating or preventing a tumor.

In other examples, the methods include culturing at least one cell including a nucleic acid molecule encoding a functional GFP (or tdT) and a homozygous null p53 mutation (such as in the absence of a NF1 mutation), which is isolated or purified from a p53 MADM transgenic mouse as described herein, contacting the cell with at least one test compound, determining the phenotype of the cell (or a population of cells), and selecting a compound that alters (for example, increases or decreases a measurable characteristic) the phenotype as compared to a control, thereby identifying a compound for treating or preventing a tumor.

In further examples, the methods include culturing at least one cell including a nucleic acid molecule encoding a functional GFP (or tdT) and a homozygous null NF1 mutation (such as in the absence of a p53 mutation), which is isolated or purified from a NF1 MADM transgenic mouse as described herein, contacting the cell with at least one test compound, determining the phenotype of the cell (or a population of cells), and selecting a compound that alters (for example, increases or decreases a measurable characteristic) the phenotype as compared to a control, thereby identifying a compound for treating or preventing a tumor.

In additional examples, the methods include administering at least one test compound to a MADM transgenic mouse described herein, determining a phenotype of the mouse, and selecting a compound that alters the phenotype (for example, increases or decreases the phenotype) of the mouse as compared to a control, thereby identifying a compound for treating or preventing a tumor (such as glioma).

In some examples, a combination of cell-based and in vivo assays may be used, for example, determining the effect of a test compound on one or more cell phenotypes in a cell-based assay, followed by determining the effect of a test compound on one or more phenotypes (such as tumor characteristics) of a p53-NF1 MADM mouse, a p53 MADM mouse, or an NF1 MADM mouse in vivo. For example, test compounds may be screened utilizing cell-based methods (such as those described below) and those that are identified as candidate compounds for treating or preventing a tumor may be screened utilizing in vivo methods (such as those described below).

A. Cell-Based Methods

Disclosed herein are cell-based methods for identifying compounds that treat or prevent a tumor (such as a glioma) utilizing cells (such as purified cells) from a transgenic mouse tumor model described herein. In some examples, the method includes identifying compounds that treat or prevent tumors, for example by contacting at least one cell (for example a cell in cell culture) from a transgenic mouse described herein with at least one test compound (such as 1, 2, 3, 4, 5, 6, 7, 8, 9, 10, or more test compounds).

In one example, the method includes culturing at least one cell which is homozygous null for p53 and NF1 and which expresses GFP (or tdT) from the disclosed p53-NF1 MADM transgenic mice, contacting the cell with at least one test compound, determining a phenotype of the cell or a population of cells (such as cell number, cell proliferation, cell cycle stage, or cell death), and selecting a compound that alters (for example, increases or decreases) the phenotype of the cell or cell population as compared to a control. In a particular example, the method further includes co-culturing the cell with at least one second cell that is wild type for p53 and NF1 and which expresses tdT (or GFP) from a p53-NF1 MADM transgenic mouse and determining a ratio of the number of first and second cells in the culture.

In another example, the method includes culturing at least one cell which is homozygous null for p53 which expresses GFP (or tdT) from the disclosed p53 MADM transgenic mice, contacting the cell with at least one test compound, determining a phenotype of the cell or a population of cells (such as cell number, cell proliferation, cell cycle stage, or cell death), and selecting a compound that alters (for example, increases or decreases) the phenotype of the cell or cell population as compared to a control. In a particular example, the method further includes co-culturing the cell with at least one second cell that is wild type for p53 and which expresses tdT (or GFP) from a p53 MADM transgenic mouse and determining a ratio of the number of first and second cells in the culture.

In another example, the method includes culturing at least one cell which is homozygous null for NF1 and which expresses GFP (or tdT) from the disclosed NF1 MADM transgenic mice, contacting the cell with at least one test compound, determining a phenotype of the cell or a population of cells (such as cell number, cell proliferation, cell cycle stage, or cell death), and selecting a compound that alters (for example, increases or decreases) the phenotype of the cell or cell population as compared to a control. In a particular example, the method further includes co-culturing the cell with at least one second cell that is wild type for NF1 and which expresses tdT (or GFP) from a NF1 MADM transgenic mouse and determining a ratio of the number of first and second cells in the culture.

In the following discussion, the methods are described with reference to a transgenic mouse wherein the cells which have undergone somatic recombination and thus are homozygous null for p53, NF1, or both p53 and NF1 and are labeled with GFP and can be identified by detecting green fluorescence. However, it is to be understood that the same methods are applicable to a transgenic mouse wherein the cells which are homozygous null for p53, NF1, or both p53 and NF1 are labeled with tdT (or another suitable fluorescent protein). In such a situation, the mutant cells can be identified by detecting red fluorescence (or other fluorescence), rather than green.

The control can be any suitable control against which to compare the phenotype of the cells. In some examples, the control is a cell or a population of cells which has not been contacted with a test compound, such as a cell from the same mouse or a different mouse. In additional examples, the control is a reference value or range of values. For example, the reference value can be derived from average phenotype values (for example cell number, cell proliferation, cell cycle stage, or cell death) obtained from a population of cells which have not been contacted with the test compound.

In some examples, cells that are homozygous null for p53, NF1, or both p53 and NF1 and that express GFP may be purified from MADM transgenic mice described herein using routine methods, such as fluorescence-activated cell sorting (FACS). In some examples, a sample from a transgenic mouse (such as a brain sample, for example, a glioma sample) may be dissociated into single cells (for example, using papain, such as Papain Dissociation System, Worthington Biochemical Corp. (Lakewood, N.J.)). The cells expressing GFP can be separated from the resulting cell mixture using FACS and collected. Preferably, a preparation of cells homozygous null for p53, NF1, or both p53 and NF1 and expressing GFP is purified such that the GFP-expressing cells represent at least 50% (for example, at least 60%, at least 70%, at least 80%, at least 85%, at least 90%, at least 95% or more) of the total cell content of the preparation. One of skill in the art can utilize other methods to isolate GFP-expressing cells, for example, antibody-based methods, such as immunopanning.

The purified GFP-expressing cells may then be placed into cell culture (for example in multi-well plates, such as 6-well, 12-well, 24-well, 96-well, or 384-well plates). The cells are maintained for a sufficient period of time to establish the culture prior to contacting with one or more test compounds (for example, about 6 hours, 12 hours, 16 hours, 24 hours, 48 hours, or more). In a particular example, the cells are cultured overnight prior to contacting with a test compound. Any suitable medium for growing the cells may be used. In a particular example, the medium is neural basal medium with B27 supplement plus 10 ng/ml PDGF, however, one of skill in the art can readily determine other suitable media (for example, based on the cell type to be cultured).

In additional examples, cells that are wild type for p53, NF1, or both p53 and NF1 and that express tdT may be isolated from the same or a different mouse using FACS or other routine techniques. A mixture of mutant and wild type cells may be placed into cell culture as a co-culture. In particular examples, the co-culture initially contains about a 1:1 ratio of mutant (e.g., green) to wild type (e.g., red) cells. In other examples, the initial ratio of mutant to wild type cells in co-culture may be about 2:1, 3:1, 5:1, 10:1, 20:1, 50:1, 100:1, or more, or about 1:2, 1:3, 1:5, 1:10, 1:20, 1:50, 1:100, or less.

Cells may be purified from the disclosed transgenic mice at a variety of time points. In some examples, the cells may be purified prior to the expected development of a tumor (such as a glioma), such as up to about 3 months post-natal (for example, at embryonic stages, at birth, or about 10 days, 2 weeks, 3 weeks, 4 weeks, 6 weeks, 8 weeks, or 12 weeks post-natal). In other examples, the cells may be purified at about the time of the expected development of a tumor. In some examples, the cells are purified at about the time of expected development of glioma, such as about 4 months post-natal (for example, about 14-18 weeks post-natal) or after the development or expected development of glioma (for example, about 20 weeks post-natal or later).

After a suitable period of time to establish the cell culture (for example, sufficient time for cells to adhere to the culture vessel or time for the cells to reach a desired percent confluency), the purified cells in culture are contacted with at least one test compound in a dose ranging from 1 nM to 1 mM (for example, about 10 nM to 100 μM, about 100 nM to 10 μM, or about 1 μM) for a suitable period of time (for example, about 12 hours, 1 day, 2 days, 3 days, 4 days, 5 days, 7 days, 10 days, 14 days, or more) and one or more cell phenotypes is then determined.

1. Cell Number

In some examples, the measured or detected cell phenotype is cell number, such as the number of cells in a cell population (for example, cells that are homozygous null for p53, NF1, or both p53 and NF1 and express GFP. In some examples, the initial cell culture includes substantially only cells that are homozygous null for p53, NF1, or both p53 and NF1 and express GFP. The culture is contacted with at least one test compound and the total number of cells may be counted after a suitable period of time (for example, after about 12 hours, 16 hours, 24 hours, 2 days, 3 days, 4 days, 5 days, 6 days, 7 days, 10 days, 14 days, or more). A decrease in total cell number in the culture (for example, a decrease of at least about 5%, at least about 10%, at least about 15%, at least about 20%, at least about 25%, at least about 30%, at least about 50%, at least about 75%, or even at least about 90%) as compared to a control (such as untreated cells cultured in parallel) identifies the compound as a compound for treating or preventing a tumor.

In other examples, the initial culture includes a mixed population of cells, such as cells that are homozygous null for p53, NF1, or both p53 and NF1 and express GFP and other cells (for example, cells that are wild type for p53, NF1, or both p53 and NF1 and express tdT and/or cells that are heterozygous for p53, NF1, or both p53 and NF1). The culture is contacted with at least one test compound and the total number of cells expressing GFP may be counted after a suitable period of time (for example, after about 12 hours, 16 hours, 24 hours, 2 days, 3 days, 4 days, 5 days, 6 days, 7 days, 10 days, 14 days, or more). Cells that express GFP may be determined by detecting fluorescence by GFP, for example by fluorescence microscopy, fluorescence plate reader, or FACS. A decrease in the number of GFP-expressing cells in the culture (for example, a decrease of at least about 5%, at least about 10%, at least about 15%, at least about 20%, at least about 25%, at least about 30%, at least about 50%, at least about 75%, or even at least about 90%) as compared to a control (such as untreated cells cultured in parallel) identifies the compound as a compound for treating or preventing a tumor.

In further examples, cells that are homozygous null for p53, NF1, or both p53 and NF1 and express GFP (mutant, green cells) and cells that are wild type for p53, NF1, or both p53 and NF1 and express tdT (wild type, red cells) are each separately purified from a sample from a transgenic mouse described herein. The two cell types are then placed in a co-culture. In some examples, the initial ratio of mutant to wild type cells in co-culture may be about 1:1, 2:1, 3:1, 5:1, 10:1, 20:1, 50:1, 100:1, or more, or about 1:2, 1:3, 1:5, 1:10, 1:20, 1:50, 1:100, or less. The co-culture is contacted with at least one test compound and a ratio of mutant to wild type cells may be determined after a suitable period of time (for example, after about 12 hours, 16 hours, 24 hours, 2 days, 3 days, 4 days, 5 days, 6 days, 7 days, 10 days, 14 days, or more), for example by detecting green fluorescence from cells expressing GFP and red fluorescence from cells expressing tdT. A decrease in the ratio of mutant cells to wild type cells (for example, a decrease of at least about 5%, at least about 10%, at least about 15%, at least about 20%, at least about 25%, at least about 30%, at least about 50%, at least about 75%, or even at least about 90%) as compared to a control (such as untreated cells cultured in parallel) identifies the compound as a compound for treating or preventing a tumor. In particular examples, the initial ratio of mutant cells to wild type cells in the co-culture is about 1:1 (for example, an approximately equal number of mutant and wild type cells are placed in the initial culture).

2. Cell Proliferation

In some examples, the measured or detected cell phenotype is cell proliferation, such as the number of cells that are dividing in the culture. The culture is contacted with at least one test compound and the number of proliferating cells may be counted after a suitable period of time (for example, after about 12 hours, 16 hours, 24 hours, 2 days, 3 days, 4 days, 5 days, 6 days, 7 days, 10 days, 14 days, or more). A decrease in the number of cells that are proliferating (such as the number of proliferating cells that are homozygous null for p53, NF1, or both p53 and NF1 and express GFP) of at least about 5%, at least about 10%, at least about 15%, at least about 20%, at least about 25%, at least about 30%, at least about 50%, at least about 75%, or even at least about 90% as compared to a control (such as untreated cells cultured in parallel) identifies the compound as a compound for treating or preventing a tumor.

Methods of determining cell proliferation are well known in the art. For example, proliferating cells may be detected by determining the expression of one or more genes that are expressed in dividing cells (cell proliferation markers). In some examples, cells that are expressing Ki67, proliferating cell nuclear antigen (PCNA), or MCM6 are detected such as by immunoassay (for example, immunohistochemistry, ELISA, or Western blot). In particular examples, the cell proliferation markers are detected by immunohistochemistry. A decrease in the number of cells that are proliferating (for example, a decrease of at least about 5%, at least about 10%, at least about 15%, at least about 20%, at least about 25%, at least about 30%, at least about 50%, at least about 75%, or even at least about 90% as compared to a control (such as untreated cells cultured in parallel) identifies the compound as a compound for treating or preventing a tumor (such as glioma). Cells that express GFP (and are homozygous null for p53, NF1, or both p53 and NF1) may be detected in the same immunohistochemistry sample, for example by fluorescence microscopy. In some examples, the cells that are proliferating are the same cells as those that express GFP. A decrease in the number of cells that are proliferating and express GFP (for example, a decrease of at least about 5%, at least about 10%, at least about 15%, at least about 20%, at least about 25%, at least about 30%, at least about 50%, at least about 75%, or even at least about 90% as compared to a control (such as untreated cells cultured in parallel) identifies the compound as a compound for treating or preventing a tumor.

In another example, proliferating cells are identified by detecting incorporation of a detectable marker (such as bromodeoxyuridine (BrdU)) into DNA of dividing cells or quantifying binding of a detectable marker to DNA (for example, CyQUANT® Direct assay, Invitrogen, Carlsbad, Calif.).

3. Cell Death

In another example, the measured or detected cell phenotype is cell death, such as the number of cells that are undergoing or have undergone apoptosis. The culture is contacted with at least one test compound and the number of dead or dying cells may be counted after a suitable period of time (for example, after about 12 hours, 16 hours, 24 hours, 2 days, 3 days, 4 days, 5 days, 6 days, 7 days, 10 days, 14 days, or more). An increase in the number of cells that are dead or dying (such as cells that express GFP and are homozygous null for p53, NF1, or both p53 and NF1) of at least about 5%, at least about 10%, at least about 15%, at least about 20%, at least about 25%, at least about 30%, at least about 50%, at least about 75%, or even at least about 90%, at least about 1.5-fold, at least about 2-fold, at least about 5-fold, at least about 10-fold, or more as compared to a control (such as untreated cells cultured in parallel) identifies the compound as a compound for treating or preventing a tumor.

Methods of measuring apoptosis are well known in the art. For example, apoptotic cell death can be characterized by cell shrinkage, membrane blebbing and chromatin condensation culminating in cell fragmentation. Cells undergoing apoptosis also display a characteristic pattern of internucleosomal DNA cleavage. Apoptosis can be measured in the presence or the absence of Fas-mediated signals. In another example, cytochrome C release from mitochondria during cell apoptosis can be detected (see, for example, Bossy-Wetzel et al., Meth. Enzymol. 322:235-42, 2000). Other assays include cytofluorometric quantitation of nuclear apoptosis induced in a cell-free system (see, for example, Lorenzo et al., Meth. Enzymol. 322:198-201, 2000), apoptotic nuclease assays (see, for example, Hughes, F M, Meth. Enzymol. 322:47-62, 2000), microscopic analysis of apoptotic cells by flow and laser scanning cytometry (see, for example, Darzynkiewicz et al., Meth. Enzymol. 322:18-39, 2000), annexin-V/propidium iodide labeling, and assays that detect DNA cleavage (see, for example, Kauffman et al., Meth. Enzymol. 322:3-15, 2000).

Apoptosis can also be measured by terminal deoxynucleotidyl transferase incorporation of labeled nucleotides into DNA strand breaks (TUNEL assay). For example, a fluorescent TUNEL assay that measures apoptotic DNA fragmentation by directly incorporating fluorescein-12-dUTP at the 3′-OH DNA ends using terminal deoxynucleotidyl transferase may be used. The fluorescein-dUTP-labeled DNAs from transfected cells are visualized directly by fluorescence microscope or quantitated by flow cytometry. Apoptosis can also be measured by determining the DNA content of cells, for example by FACS analysis of cells stained with a fluorescent DNA dye. Cells having less than 2N DNA content are cells which are undergoing apoptosis.

4. Tumor Sphere Assay

In another example, the measured or detected cell phenotype is a tumor sphere characteristic for example, the number or size of tumor spheres, differentiation of tumor sphere cells, or tumorigenic ability of tumor spheres. A “tumor sphere” is a mass of tumor cells that can form upon culture of dissociated tumor cells (for example glioma cells). Without being bound by theory, it is believed that tumor spheres are formed from tumor stem cells (see, e.g. Yuan et al., Oncogene 23:9392-9400, 2004; Sutter et al., Biochim. Biophys. Acta 1776:125-137, 2007; Colleoni and Torrente, Cancer Lett. 272:1-11, 2008). In addition to the ability to form tumor spheres, tumor stem cells exhibit capacity for self-renewal (the ability to reform a tumor sphere following dissociation), expression of stem cell markers (for example, neural stem cell markers), the ability to differentiate into mature cell types (such as neurons, astrocytes, and oligodendrocytes), and the ability to form tumors after implantation into a mouse. These characteristics of tumor stem cells can be utilized to identify a compound for treating or preventing a tumor (such as glioma), such as a compound that inhibits one or more of these characteristics of tumor stem cells present in a tumor.

In some examples, a tumor (such as glioma) is isolated from a MADM transgenic mouse disclosed herein and a culture of dissociated cells from the tumor is established. In particular examples, the culture is contacted with at least one test compound and the number of tumor spheres formed is counted after a suitable period of time (for example, after about 12 hours, 16 hours, 24 hours, 2 days, 3 days, 4 days, 5 days, 6 days, 7 days, 10 days, 14 days, or more). A decrease in the number of tumor spheres of at least about 5%, at least about 10%, at least about 15%, at least about 20%, at least about 25%, at least about 30%, at least about 50%, at least about 75%, or even at least about 90%, or more as compared to a control (such as tumor cells cultured in parallel but not contacted with the compound) identifies the compound as a compound for treating or preventing a tumor.

In other examples, a culture of dissociated cells from a tumor isolated from a disclosed MADM transgenic mouse is contacted with at least one test compound and the size of one or more tumor spheres is determined (such as sphere diameter or number of cells in the sphere, or the average of a population of tumor spheres) after a suitable period of time (for example, after about 12 hours, 16 hours, 24 hours, 2 days, 3 days, 4 days, 5 days, 6 days, 7 days, 10 days, 14 days, or more). A decrease in the size of tumor spheres of at least about 5%, at least about 10%, at least about 15%, at least about 20%, at least about 25%, at least about 30%, at least about 50%, at least about 75%, or even at least about 90%, or more as compared to a control (such as tumor cells cultured in parallel but not contacted with the compound) identifies the compound as a compound for treating or preventing a tumor.

In another example, one or more tumor spheres are established from a tumor isolated from a disclosed MADM transgenic mouse disclosed herein. The tumor spheres are contacted with at least one test compound for a suitable period of time (for example, after about 12 hours, 16 hours, 24 hours, 2 days, 3 days, 4 days, 5 days, 6 days, 7 days, 10 days, 14 days, or more). The tumor spheres are then dissociated and placed into a new culture. The formation of new tumor spheres (“self-renewal”) is determined (for example, the number or size of new tumor spheres or the efficiency of formation of new tumor spheres). A decrease in the number and/or size of tumor spheres or the efficiency of their formation, such as a decrease of at least about 5%, at least about 10%, at least about 15%, at least about 20%, at least about 25%, at least about 30%, at least about 50%, at least about 75%, or even at least about 90%, or more as compared to a control (such as tumor spheres cultured in parallel but not contacted with the compound) identifies the compound as a compound for treating or preventing a tumor.

In some examples, one or more tumor spheres are established from a tumor isolated from a p53, NF1, or p53-NF1 MADM transgenic mouse disclosed herein. The tumor spheres are contacted with at least one test compound for a suitable period of time (for example, after about 12 hours, 16 hours, 24 hours, 2 days, 3 days, 4 days, 5 days, 6 days, 7 days, 10 days, 14 days, or more). The tumor spheres are then exposed to suitable medium (such as medium containing about 1% fetal bovine serum) for a period of time (such as 1-7 days, for example 3-5 days), then assessed for differentiation to mature cell types (for example, neurons, astrocytes, and oligodendrocytes if the tumor is a glioma). Markers for identifying differentiated cell types are well known to one of skill in the art. In some examples, the markers include, but are not limited to GFAP for astrocytes, microtubule-associated protein 2 (MAP2) for neurons, and O4 for oligodendrocytes. An increase in the number of cells expressing a marker of differentiation and/or the amount of expression of such markers, for example, at least about 5%, at least about 10%, at least about 15%, at least about 20%, at least about 25%, at least about 30%, at least about 50%, at least about 75%, at least about 90%, at least about 1.5-fold, at least about 2-fold, at least about 5-fold, at least about 10-fold, or more as compared to a control (such as tumor spheres cultured in parallel but not contacted with the compound) identifies the compound as a compound for treating or preventing a tumor.

The tumor spheres may also be assessed for expression of stem cell markers, (such as nestin and Sox2 if the tumor is a glioma). A decrease in the number of cells expressing a stem cell marker and/or the amount of expression of such markers, for example, at least about 5%, at least about 10%, at least about 15%, at least about 20%, at least about 25%, at least about 30%, at least about 50%, at least about 75%, at least about 90%, or more as compared to a control (such as tumor cells cultured in parallel but not contacted with the compound) identifies the compound as a compound for treating or preventing a tumor.

In further examples, one or more tumor spheres are established from a tumor isolated from a MADM transgenic mouse disclosed herein. The tumor spheres are contacted with at least one test compound for a suitable period of time (for example, after about 12 hours, 16 hours, 24 hours, 2 days, 3 days, 4 days, 5 days, 6 days, 7 days, 10 days, 14 days, or more). The tumor spheres are transplanted into a mouse (such as an immune-compromised mouse, for example, a SCID mouse) and the formation of tumors is determined. A decrease in the number and/or size of tumors in the mouse, such as at least about 5%, at least about 10%, at least about 15%, at least about 20%, at least about 25%, at least about 30%, at least about 50%, at least about 75%, or even at least about 90%, or more as compared to a control (such as tumor spheres cultured in parallel but not contacted with the compound) identifies the compound as a compound for treating or preventing a tumor.

5. Cell Differentiation

In another example, the measured or detected cell phenotype is cell differentiation, such as the number of cells that express molecular markers of differentiated cells (such as oligodendrocytes) or the number of cells exhibiting morphological characteristics of differentiated cells (such as oligodendrocytes). In some examples, differentiated cells are also identified by a decrease in expression of tumor markers.

In some examples, a tumor (such as glioma) is isolated from a MADM transgenic mouse disclosed herein and a culture of dissociated cells from the tumor is established. The culture is contacted with at least one test compound and the number of differentiated cells may be counted after a suitable period of time (for example, after about 12 hours, 16 hours, 24 hours, 2 days, 3 days, 4 days, 5 days, 6 days, 7 days, 10 days, 14 days, or more). In some examples, an increase in the number of cells that are differentiated (such as cells that express molecular markers of differentiated cells or cells exhibiting morphological characteristics of differentiated cells) of at least about 5%, at least about 10%, at least about 15%, at least about 20%, at least about 25%, at least about 30%, at least about 50%, at least about 75%, or even at least about 90%, at least about 1.5-fold, at least about 2-fold, at least about 5-fold, at least about 10-fold, or more as compared to a control (such as untreated cells cultured in parallel) identifies the compound as a compound for treating or preventing a tumor. In other examples, a decrease in the number of undifferentiated cells (such as cells that express tumor cell markers or cells that exhibit morphological characteristics of undifferentiated tumor cells) of at least about 5%, at least about 10%, at least about 15%, at least about 20%, at least about 25%, at least about 30%, at least about 50%, at least about 75%, at least about 90%, or more as compared to a control (such as tumor cells cultured in parallel but not contacted with the compound) identifies the compound as a compound for treating or preventing a tumor. In some examples, molecular markers of differentiated cells include CD82, O1, PLP, myelin basic protein (MBP), myelin oligodendrocyte glycoprotein (MOG), and myelin-associated glycoprotein (MAG). In other examples, tumor cell markers include NG2, PDGFRα, CD9, and O4.

Methods of detecting gene expression (such as genes that are markers for tumor cells or differentiated cells) are well known in the art. For example, gene expression can be evaluated by detecting mRNA encoding the gene of interest. RNA can be isolated from a sample (for example, a tumor sample) from a transgenic mouse, a sample of adjacent non-tumor tissue from the transgenic mouse, a sample (for example, tumor or non-tumor tissue) from an untreated mouse, or combinations thereof, using methods well known to one skilled in the art, including commercially available kits. In some examples, mRNA expression in a sample is quantified using PCR-based methods, such as reverse transcription polymerase chain reaction (RT-PCR) (Weis et al., Trends Genet. 8:263-264, 1992) or microarray techniques.

In other examples, in situ hybridization (ISH) is utilized for detecting and comparing expression of genes of interest. ISH applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, and allows the localization of sequences to specific cells within populations, such as tumor and non-tumor cells or mutant and wild type cells.

In other examples, gene expression of tumor markers or markers of cell differentiation can be evaluated by detecting one or more proteins encoded by the genes of interest. Antibodies specific for the proteins of interest can be used for detection and quantitation of proteins by one of a number of immunoassay methods that are well known in the art, such as those presented in Harlow and Lane (Antibodies, A Laboratory Manual, CSHL, New York, 1988). Methods of constructing such antibodies are known in the art. In addition, such antibodies may be commercially available. Any standard immunoassay format (such as ELISA, Western blot, or RIA assay) can be used to measure protein levels. Immunohistochemical techniques can also be utilized for protein detection and quantification. General guidance regarding such techniques can be found in Bancroft and Stevens (Theory and Practice of Histological Techniques, Churchill Livingstone, 1982) and Ausubel et al. (Current Protocols in Molecular Biology, John Wiley & Sons, New York, 1998).

One of skill in the art can also identify differentiated cells based on their morphological characteristics. In some examples, differentiated cells include cells that have a rounded phenotype with elaborated processes, while undifferentiated (e.g., tumor) cells include spindle-shaped cells. See, e.g., Gobert et al., Mol. Cell. Biol. 29:1538-1553, 2009.

B. In Vivo Methods

Disclosed are methods for identifying compounds that treat or prevent a tumor utilizing the transgenic mice described herein. In some examples, the methods include identifying compounds that treat or prevent a tumor (such as glioma) in vivo, for example by administering at least one test compound (such as 1, 2, 3, 4, 5, 6, 7, 8, 9, 10, or more test compounds) to a transgenic mouse that is mosaic for homozygous p53 and NF1 null mutations, a transgenic mouse that is mosaic for a homozygous p53 null mutation, or a transgenic mouse that is mosaic for a homozygous NF1 null mutation.

In one embodiment, the method includes administering at least one test compound to a transgenic mouse of the disclosure, determining a phenotype of the transgenic mouse (such as the number of GFP and/or tdT expressing cells, tumor characteristics, or gene expression), and selecting a compound that alters (for example, increases or decreases) the phenotype as compared to a control.

In the following discussion, the methods are described with reference to a transgenic mouse wherein the cells which have undergone somatic recombination and thus are homozygous null for both p53 and NF1, cells which have undergone somatic recombination and are homozygous null for p53, or cells which have undergone somatic recombination and are homozygous null for NF1 are labeled with GFP and can be identified by detecting green fluorescence. However, it is to be understood that the same methods are applicable to a transgenic mouse wherein the cells which are homozygous null for both p53 and NF1, homozygous null for p53, or homozygous null for NF1 are labeled with tdT (or another suitable fluorescent protein). In such a situation, the mutant cells can be identified by detecting red fluorescence (or other fluorescence), rather than green.

The control can be any suitable control against which to compare the phenotype of the transgenic mouse. In some examples, the control is a transgenic mouse to which the at least one test compound has not been administered. In other examples, the control is from the same transgenic mouse to which at least one test compound has been administered, such as a phenotype of the mouse prior to administering the at least one test compound. In additional examples, the control is a reference value or ranges of values. For example, the reference value can be derived from average phenotype values (for example, the number of cells expressing GFP and/or tdT, tumor size, or tumor number) obtained from a group of transgenic mice to which the test compound has not been administered.

1. Fluorescence

In one example, the measured or detected phenotype is the number of mutant cells (those homozygous for p53 and NF1 knockout alleles or homozygous for p53 knockout alleles, or those homozygous for NF1 knockout alleles, for example, those expressing GFP) measured in a sample (such as a tumor sample, for example, a glioma sample) from a transgenic mouse to which at least one test compound has been administered. A decrease in the number of mutant cells (for example, green cells) in the presence of a test compound (for example, a decrease of at least about 5%, at least about 10%, at least about 15%, at least about 20%, at least about 25%, at least about 30%, at least about 50%, at least about 75%, at least about 90%, at least about 95%, or even more) as compared to a control identifies the compound as a compound for treating or preventing a tumor.

In another example, the ratio of mutant (e.g., green) cells to wild type (e.g., red) cells is measured in a sample (such as a tumor sample, for example, a glioma sample) from a transgenic mouse to which at least one test compound has been administered. A decrease in the ratio of mutant cells to wild type cells in a sample from a transgenic mouse that has been administered at least one test compound (for example, a decrease of at least about 5%, at least about 10%, at least about 15%, at least about 20%, at least about 25%, at least about 30%, at least about 50%, at least about 75%, at least about 90%, at least about 95%, or even more) as compared to a control indicates that the compound is a compound for treating or preventing a tumor.

Methods of detecting the labeled mutant (e.g., green) cells and wild type (e.g., red) cells (or vice versa) are known to one of skill in the art. In some examples, the labeled cells are detected using fluorescence, such as microscopy (for example, wide field microscopy or confocal microscopy) or with a fluorescence reader (such as a microplate reader or FACS analyzer). Appropriate excitation-emission wavelengths are well known to one of skill in the art. Exemplary excitation-emission data for GFP and RFP (such as tdT) and related proteins are presented in Table 2.

TABLE 2
Exemplary excitation-emission data
for selected fluorescent proteins

| Protein | Excitation max (nm) | Emission max (nm) |
|---|---|---|
| Wild type GFP | 395 | 509 |
| GFP S65T | 489 | 509 |
| EGFP | 488 | 509 |
| DsRed | 558 | 583 |
| mRFP1 | 584 | 607 |
| tdTomato | 554 | 581 |
| mCherry | 587 | 610 |

In some examples, a tissue section (such as a brain section, for example, a sagittal brain section) may be obtained from a transgenic mouse described herein and prepared for fluorescence microscopy. In other examples, a tumor sample (for example, a glioma) may be isolated from a transgenic mouse described herein. The tumor sample may be sectioned and prepared for fluorescence microscopy, or the tumor cells may be dissociated and fluorescence may be measured using a fluorescent plate reader. In some examples, the labeled cells are detected by direct fluorescence (for example, live imaging). In other examples, the labeled cells are detected by indirect fluorescence. If indirect fluorescence is utilized, the tissue section or dissociated cells are incubated with a primary antibody that recognizes the fluorescent protein (such as GFP or tdT), followed by a secondary antibody that is labeled with a fluorescent tag (such as FITC or Cy3).

2. Tumor Characteristics

In some examples, the measured or detected phenotype is a tumor characteristic such as tumor size (for example, tumor volume or tumor cell number), tumor number (for example, number of primary tumors or number of tumor metastases), occurrence or number of tumor metastases or tumor recurrence in a disclosed MADM transgenic mice.

In a particular example, tumor size is assessed in a transgenic mouse that is administered at least one test compound. In some examples, a decrease in tumor size (such as tumor volume or tumor cell number) in a transgenic mouse administered a test compound (for example, a decrease of at least about 5%, at least about 10%, at least about 15%, at least about 20%, at least about 25%, at least about 30%, at least about 50%, at least about 75%, or even at least about 90%) as compared to a control indicates that the compound is a compound for treating or preventing a tumor.

Methods of measuring tumor size in a mouse are well known to one of skill in the art. For example, tumor size (such as tumor volume) may be measured using in vivo imaging (for example, magnetic resonance imaging, computed tomography, or positron emission tomography). Tumor size may also be measured by measuring one or more tumor dimensions, such as length, width, or diameter (for example, in a tissue section from an animal or an isolated tumor). In other examples, tumor cell number may be assessed, for example by isolating a tumor (such as a glioma) from an animal and counting the number of cells present in the tumor or in a defined volume of the tumor.

In another example, tumor number is assessed in a transgenic mouse that is administered at least one test compound. A decrease in the number of tumors (such as gliomas) in a transgenic mouse administered a test compound (for example, a decrease of at least about 5%, at least about 10%, at least about 15%, at least about 20%, at least about 25%, at least about 30%, at least about 50%, at least about 75%, or even at least about 90%) as compared to a control identifies the compound as a compound for treating or preventing glioma. The number of tumors in a transgenic mouse may be counted by in vivo imaging (for example, magnetic resonance imaging or computed tomography) or by histological or immunohistochemical examination of a sample from a transgenic mouse. Tumor pathology can be identified by one of skill in the art. For example, hallmarks of glioma pathology include pseudopalisading necrosis (areas of necrosis surrounded by anaplastic cells), hyperplastic vasculature, peri-neuronal satellitosis, and increased mitotic index.

In other examples, tumor recurrence is assessed in a transgenic mouse that is administered at least one test compound. A decrease in the recurrence of a tumor (such as glioma) in a transgenic mouse administered at least one test compound (for example, a decrease of at least about 5%, at least about 10%, at least about 15%, at least about 20%, at least about 25%, at least about 30%, at least about 50%, at least about 75%, or even at least about 90%) as compared to a control identifies the compound as a compound for treating or preventing a tumor. Recurrence of a tumor is the return of a tumor (either in the same location or the same tissue as the primary tumor) after the tumor has been removed surgically, by drug or other treatment (for example, following administration of a test compound as described herein), or has otherwise disappeared. Recurrences may be detected by in vivo imaging (for example, magnetic resonance imaging or computed tomography) or by histological or immunohistochemical examination of a sample from a transgenic mouse.

In a further example, a decrease in the metastasis of a tumor (for example, the number or aggressiveness of metastases) in a transgenic mouse administered at least one test compound (for example, a decrease of at least about 5%, at least about 10%, at least about 15%, at least about 20%, at least about 25%, at least about 30%, at least about 50%, at least about 75%, or even at least about 90%) as compared to a control identifies the compound as a compound for treating or preventing a tumor. Metastasis of glioma is rare, however metastasis to the meninges, lymph nodes (for example, cervical or mediastinal lymph nodes), or other organs (for example, lung, liver, or bone) does occur. In some examples, aggressiveness of glioma metastasis includes metastasis outside of the central nervous system (CNS). Thus, a decrease in aggressiveness of metastasis includes a decrease in the number of metastases outside of the CNS.

In further examples, the phenotype is delay of the development, progression, or metastasis of a tumor. For example, a delay in development, progression or metastasis by at least about 1 month, 2 months, 3 months, 4 months, 5, months, 6 months, one year, or more in a transgenic mouse administered at least one test compound as compared to a control identifies the compound as a compound for treating or preventing glioma.

3. Gene Expression

In some examples, the measured or detected phenotype is the expression of genes that are markers for a tumor (for example, GFAP, Sox2, NG2, PDGFRα, Nestin, or Olig2 in glioma) or markers of cell proliferation (such as Ki67, PCNA, or MCM6) by cells (such as glial cells) of a disclosed MADM transgenic mouse. A decrease in expression of tumor cell markers or cell proliferation markers in a sample (such as tumor tissue) from a transgenic mouse administered at least one test compound (for example, a decrease of at least about 5%, at least about 10%, at least about 15%, at least about 20%, at least about 25%, at least about 30%, at least about 50%, at least about 75%, or even at least about 90%) as compared to a control identifies the compound as a compound for treating or preventing a tumor.

Methods of detecting gene expression (such as genes that are markers for tumor cells or cell proliferation) are well known in the art. Exemplary methods are discussed in Section A, above). In some non-limiting examples, tumor markers or markers of cell proliferation include GFAP, Sox2, NG2, PDGFRα, Nestin, Olig2, and Ki67.

4. Morbidity and Mortality

In some examples, the measured or detected phenotype is mortality of a disclosed MADM transgenic mouse. A decrease in mortality (such as an increased survival time) in a transgenic mouse administered at least one test compound (for example, a decrease of at least about 5%, at least about 10%, at least about 15%, at least about 20%, at least about 25%, at least about 30%, at least about 50%, at least about 75%, or even at least about 90%) as compared to a control identifies the compound as a compound for treating or preventing a tumor. For example, a decrease in mortality can be determined by following one or more p53-NF1 MADM, p53 MADM, or NF1 MADM transgenic mice to which at least one test compound has been administered until death occurs. A decrease in mortality may include an increased age at death as compared to a control.

In other examples, the measured or detected phenotype is morbidity of the disclosed p53-NF1 MADM, p53 MADM, or NF1 MADM transgenic mice. A decrease the number or severity of symptoms of a tumor (for example, lethargy, weight loss, hunched posture, poor grooming, ataxia, hyperactivity, or hydrocephaly) in a transgenic mouse administered at least one test compound as compared to a control identifies the compound as a compound for treating or preventing a tumor.

5. Administration of Test Compounds

Test compounds are administered to the disclosed transgenic mouse in any suitable manner, preferably with pharmaceutically acceptable carriers. Pharmaceutically acceptable carriers are determined in part by the particular composition being administered, as well as by the particular method used to administer the composition. Accordingly, there is a wide variety of suitable formulations of pharmaceutical compositions of the present disclosure. The pharmaceutically acceptable carriers useful in this disclosure are conventional. Remington: The Science and Practice of Pharmacy, ed., Hendrickson, Lippincott Williams & Wilkins, Philadelphia, Pa., 21^{st} Edition (2005), describes compositions and formulations suitable for pharmaceutical delivery of test compounds.

In some examples one or more test compounds (such as one, two, three, four, five, ten, or twenty) are administered to a mouse. In some embodiments, the one or more test compounds are administered to a mouse in a single dose. In other embodiments, the one or more test compounds are administered to a mouse in multiple doses. When administered in multiple doses, the time period between each administration can vary and will depend in part on the type of tumor involved. In some examples, the one or more test compounds are administered daily, bi-weekly, weekly, bi-monthly or monthly. When administered in multiple doses, the time period between each administration can vary and will depend in part on the subject being treated and the type of tumor being treated. One of skill in the art can determine an appropriate dosing schedule for a tumor or to be used as a screening test for a compound for treating or preventing a tumor. In some examples, the one or more test compounds are administered in a dose ranging from 0.1 ng/kg to 100 mg/kg (for example, about 1 ng/kg to 10 mg/kg, about 10 ng/kg to 1 mg/kg, or about 100 ng/kg).

### V. Test Compounds

The methods disclosed herein are of use for identifying compounds that can be used to treat or prevent a tumor (such as glioma). A “compound” or “test compound” is any substance or any combination of substances that is useful for achieving an end or result. Any compound that has potential (whether or not ultimately realized) to treat or prevent cancer, for example by altering a transformed or pre-transformed phenotype (or other phenotype of a tumor cell) can be tested (screened) using the methods of this disclosure.

Exemplary compounds include, but are not limited to, peptides, such as soluble peptides, including but not limited to members of random peptide libraries (see, e.g., Lam et al., Nature, 354:82-84, 1991; Houghten et al., Nature, 354:84-86, 1991), and combinatorial chemistry-derived molecular libraries made of D- and/or L-configuration amino acids, phosphopeptides (including, but not limited to, members of random or partially degenerate, directed phosphopeptide libraries; see, e.g., Songyang et al., Cell, 72:767-778, 1993), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), small organic or inorganic molecules (such as, so-called natural products or members of chemical combinatorial libraries), molecular complexes (such as protein complexes), or nucleic acids (such as antisense compounds).

Appropriate compounds can be contained in libraries, for example, synthetic or natural compounds in a combinatorial library. Numerous libraries are commercially available or can be readily produced; means for random and directed synthesis of a wide variety of organic compounds and biomolecules, including expression of randomized oligonucleotides, such as antisense oligonucleotides and oligopeptides, also are known. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Such libraries are useful for the screening of a large number of different compounds.

Libraries (such as combinatorial chemical libraries) useful in the disclosed methods include, but are not limited to, peptide libraries (see, e.g., U.S. Pat. No. 5,010,175; Furka, Int. J. Pept. Prot. Res., 37:487-493, 1991; Houghton et al., Nature, 354:84-88, 1991; PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Natl. Acad. Sci. USA, 90:6909-6913, 1993), vinylogous polypeptides (Hagihara et al., J. Am. Chem. Soc., 114:6568, 1992), nonpeptidal peptidomimetics with glucose scaffolding (Hirschmann et al., J. Am. Chem. Soc., 114:9217-9218, 1992), analogous organic syntheses of small compound libraries (Chen et al., J. Am. Chem. Soc., 116:2661, 1994), oligocarbamates (Cho et al., Science, 261:1303, 1003), and/or peptidyl phosphonates (Campbell et al., J. Org. Chem., 59:658, 1994), nucleic acid libraries (see Sambrook et al. Molecular Cloning, A Laboratory Manual, Cold Springs Harbor Press, N.Y., 1989; Ausubel et al., Current Protocols in Molecular Biology, Green Publishing Associates and Wiley Interscience, N.Y., 1989), peptide nucleic acid libraries (see, e.g., U.S. Pat. No. 5,539,083), antibody libraries (see, e.g., Vaughn et al., Nat. Biotechnol., 14:309-314, 1996; PCT Publication No. WO 97/00271), carbohydrate libraries (see, e.g., Liang et al., Science, 274:1520-1522, 1996; U.S. Pat. No. 5,593,853), small organic molecule libraries (see, e.g., benzodiazepines, Baum, C&EN, January 18, page 33, 1993; isoprenoids, U.S. Pat. No. 5,569,588; thiazolidionones and methathiazones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514) and the like.

Libraries useful for the disclosed screening methods can be produced in a variety of manners including, but not limited to, spatially arrayed multipin peptide synthesis (Geysen, et al., Proc. Natl. Acad. Sci., 81(13):3998-4002, 1984), “tea bag” peptide synthesis (Houghten, Proc. Natl. Acad. Sci., 82(15):5131-5135, 1985), phage display (Scott and Smith, Science, 249:386-390, 1990), spot or disc synthesis (Dittrich et al., Bioorg. Med. Chem. Lett., 8(17):2351-2356, 1998), or split and mix solid phase synthesis on beads (Furka et al., Int. J. Pept. Protein Res., 37(6):487-493, 1991; Lam et al., Chem. Rev., 97(2):411-448, 1997). Libraries may include a varying number of compositions (members), such as up to about 100 members, such as up to about 1000 members, such as up to about 5000 members, such as up to about 10,000 members, such as up to about 100,000 members, such as up to about 500,000 members, or even more than 500,000 members.

In one convenient embodiment, high throughput screening methods involve providing a combinatorial (chemical or peptide) library containing a large number of potential therapeutic compounds. Such combinatorial libraries are then screened in one or more assays as described herein to identify those library members (particularly chemical species or subclasses) that display a desired characteristic activity (such as altering a transformed or pre-transformed cell phenotype, for example, inhibiting or decreasing tumor cell proliferation).

The compounds identified using the methods disclosed herein can serve as conventional “lead compounds” or can themselves be used as potential or actual therapeutics. In some instances, pools of candidate agents may be identified and further screened to determine which individual or subpools of agents in the collective have a desired activity.

In some examples, the test compounds can include compounds that are known or predicted to be candidates for treating glioma. In particular examples, the test compounds may include inhibitors of PDGFR, for example, imatinib, CP-673,451, vatalanib (PTK787), and nilotinib (AMN107). In other examples, the test compounds may include inhibitors of signaling molecules downstream of receptor tyrosine kinases (such as PDGFR), for example, inhibitors of Akt (such as UCN-01 or SH-5) or inhibitors of mTor (such as rapamycin or CCI-779). In further examples, the test compounds may include cytokines (for example, interleukin-6) or inhibitors of epigenetic regulation, such as inhibitors of histone deacetylase, DNA methyltransferase, or histone-lysine N-methyltransferase (for example, trichostatin A, decitabine, or 3-deazaneplanocin A).

The present disclosure is illustrated by the following non-limiting Examples.

### Example 1

### Generation of Chromosome 11 MADM System

This example describes generation of chromosome 11 MADM mouse lines.

MADM cassettes consisting of split green fluorescent protein (GFP) and red fluorescent protein tdTomato (tdT; Shaner et al., Nature Biotechnol. 22:1567-1572, 2004) were constructed. One MADM cassette included the amino terminal portion of GFP and the carboxyl terminal portion of tdT (N-GFP_loxP_C-tdT; “GT cassette”) and the other MADM cassette included the amino terminal portion of tdT and the carboxyl terminal portion of GFP (N-tdT_loxP_C-GFP, “TG cassette”). The GFP/tdT fragments in each cassette were separated by a β-globin intron including a loxP site that is recognized by Cre recombinase. The N-GFP portion encoded amino acids 1-95 of mut4-EGFP and the C-GFP portion encoded amino acids 96-239 of mut4-EGFP. The N-tdT portion encoded the first amino acid plus one nucleotide (ATGG) of tdT and the C-tdT portion encoded the remainder of the tdT protein (amino acids 2-476). The intron including the loxP site had the following sequence:

(SEQ ID NO: 1)
gttggtatcaaagatctATAACTTCGTATAGCATACATTATACGAAGTTA
Tggttacaagacaggtttaaggagaccaatagaaactgggcatgtggaga
cagagaagactcttgggtttctgataggcactgactctctctgcctattg
gtctattttcccacccttag

where lower case sequence is the intron sequence, lower case and bold sequence is a BglII site, and upper case sequence is the loxP site.

The Ensembl genome browser was used to choose an intergenic region on chromosome 11 between the Eif4enif1 and Drg1 genes as the insertion site. These genes are both housekeeping genes which are expressed at high level in almost all tissues and are therefore predicted to be in a region of open chromatin structure. They are located approximately 3-4 cM from the centromere of chromosome 11. To avoid essential non-coding DNA elements (such as those required for chromatin structure or transcriptional control), the VISTA program (available on the World Wide Web at genome.lbl.gov/vista) was used to compare the selected intergenic region between human and mouse. Non-coding regions that were conserved between mouse and human were avoided for the insertion locus. The insertion site was also chosen to avoid repetitive DNA elements in the intergenic region. Finally, a region of balanced GCAT composition was selected to avoid possible secondary structures that might interfere with the insertion of the knock-in constructs.

MADM mice carrying either the GT or the TG cassette were generated via homologous recombination technology in mouse embryonic stem (ES) cells. Specifically, “targeting arms” from chromosome 11 were obtained by PCR with mouse genomic DNA. The sequences of the 5′ and 3′ targeting arms are provided in the Sequence Listing as SEQ ID NOS: 2 and 3, respectively. The MADM cassettes (GT and TG) were inserted into the arms, respectively, with routine molecular cloning techniques. These DNA constructs then were each separately electroporated into mouse ES cells (R1 cells), and targeted clones of ES cells were identified by PCR and Southern blot. Individual targeted ES clones were expanded, and then injected into wild type blastocysts to produce chimeric mice. Finally, the chimeric mice were bred to C57BL/6 mice to obtain germline transmission of either the GT or TG construct.

Mouse strains are named using the following nomenclature system. A “,” is used to indicate two genes on the same chromosome (in cis). A “I” is used to designate genes on separate homologous chromosomes (in trans). Finally, “;” is used to separate genes that are not linked to the preceding chromosome. In an example, TG, p53KO, NF1FLOX/GT; Cre indicates that TG, p53 knockout, and floxed NF1 are on the same chromosome 11 (in cis); GT is on the other chromosome 11 (in trans); and Cre is an independent transgene not linked to chromosome 11.

To verify the successful establishment of the chromosome 11 MADM system, Chr11^{GT}/Chr11^{WT} mice were crossed with a mouse line expressing Cre from an Hprt promoter. The resulting Chr11^{GT}/Chr11^{WT};Cre mice were then crossed with Chr11^{TG}/Chr11^{WT} mice to generate Chr11^{GT}/Chr11^{TG};Cre offspring (GT/TG; Cre mice) and Chr11^{GT}/Chr11^{TG} control offspring (GT/TG control mice). In order to confirm that there was no leaky expression of the GT or TG alleles in the absence of Cre, the GT/TG control mice were examined for the presence of green or red cells. No red or green cells were present in the five GT/TG control mice examined, indicating that the recombination was completely Cre-dependent.

GT/TG; Cre mice exhibited unambiguous green, red, or double-labeled (yellow) cells in all tissues examined, including heart, kidney, cerebellum, and choroid plexus. To estimate the recombination efficiency, multiple age-matched MADM labeled brains were compared between the current targeting locus and a previously developed MADM system (Zong et al., Cell 121:479-492, 2005). The current chromosome 11 MADM system had a significantly higher recombination rate than the previous MADM system, based on the number of labeled cells in the brain.

### Example 2

### Generation of p53 and NF1 Knockout MADM Mouse Line

This example describes the generation of a mouse strain carrying p53 and NF1 knockout alleles with the chromosome 11 MADM system.

p53 is a tumor suppressor gene (TSG) that is mutated in over 50% of human cancers. p53 is implicated in brain tumors, such as glioma. Patients with Li Fraumeni syndrome resulting from germline mutation of one allele of p53 are predisposed to develop brain tumors. Further, approximately 30% of both benign astrocytoma and glioblastoma have p53 mutations and LOH of p53 is required for the malignancy. Neurofibromatosis type 1 (NF1) is associated with increased central nervous system tumors, including glioma, and approximately 15-25% of gliomas show NF1 mutations. Although NF1 knockout mice do not exhibit glioma, simultaneous knockout of NF1 and p53 resulted in development of gliomas at six months of age with almost 100% penetrance (Reilly et al., Nat. Genet. 26:109-113, 2000; Zhu et al., Cancer Cell 8:119-130, 2005). As NF1 and p53 are both located on mouse chromosome 11, the MADM system described in Example 1 was used to develop an in vivo model of LOH for both genes.

p53 knockout (p53KO) and floxed NF1 (NF1FLOX) alleles were introduced onto the TG chromosome described in Example 1 through standard breeding with selection for recombinant progeny having TG, p53KO, and NF1FLOX alleles on the same chromosome (TG, p53KO, NF1FLOX). p53KO mice were obtained from Jackson Laboratory (Bar Harbor, Me.; stock number 002101) and NF1FLOX mice were obtained from the National Cancer Institute Mouse Repository (Frederick, Md.; stock number 01XM4). First, p53KO and NF1FLOX mice were crossed to obtain p53KO/NF1FLOX (in trans) mice. Next the p53KO/NF1FLOX mice were bred to wild type mice. Heterozygous p53KO, NF1 FLOX (in cis) mice were obtained as a result of germline recombination between the p53 and NF1 loci. p53KO, NF1FLOX mice were then crossed with the TG mice described in Example 1 to generate TG/p53KO, NF1FLOX mice. These mice were subsequently crossed with wild type mice and progeny with TG, p53KO, NF1FLOX (in cis) genotype resulting from germline recombination were obtained.

Separately, the GT mice described in Example 1 were crossed with a mouse line expressing Cre recombinase under the control of a GFAP promoter (GFAP-Cre; Zhuo et al. Genesis 31:85-94, 2001) or a nestin promoter to generate GT; Cre mice. Finally the TG, p53KO, NF1FLOX and GT; Cre strains were crossed and mice with the genotype TG, p53KO, NF1FLOX/GT; Cre were selected by PCR genotyping. These mice are referred to as “p53-NF1 MADM mice.”

### Example 3

### Characterization of p53-NF1 MADM Mouse Model

This example describes the phenotype of the p53-NF1 MADM mouse generated in Example 2.

Brains from p53-NF1 MADM mice generated as described in Example 2 were harvested from mice at post-natal day 10 (P10), 2 months, and 4 months of age. Tissue sections were prepared and analyzed by fluorescence microscopy to detect cells expressing GFP (green), TdT (red), or both (yellow). For histology, mice were anesthetized with ketamine/xylazine (60 mg/kg and 8 mg/kg, respectively) and perfused with 4% paraformaldehyde (PFA) in 0.1 M phosphate buffer saline (PBS). Tissues were isolated and fixed in 4% PFA at 4° C. overnight, washed 3 times in PBS, cryoprotected for 24 hrs in 30% sucrose in PBS, and embedded in OCT prior to cryostat sectioning. Tissues were sectioned at 25 μm thickness unless otherwise specified. Images were taken with an Olympus Fluoview 1000 confocal laser scanning microscope.

Green cells (mutant) were those that have undergone somatic recombination at the loxP sites and X segregation; they expressed reconstituted GFP and were homozygous null for p53 and NF1. Red cells (wild type) were the siblings to the green cells that have undergone somatic recombination at the loxP sites and X segregation; they expressed reconstituted TdT and were wild type for both p53 and NH. Yellow cells were those that have undergone somatic recombination at the loxP sites and Z segregation; they expressed both GFP and TdT and were heterozygous null for both p53 and NF1. Proliferating cells were identified by staining with a Ki67 antibody (Cat. No. NCL-Ki67p; Leica Microsystems, Berlin, Germany) or with 5-bromo-2-deoxyuridine (BrdU) labeling. To detect rapidly dividing cells, mice were injected intraperitoneally with BrdU (40-60 mg/kg) 1.5 hours prior to dissection. To detect more slowly dividing cells, mice were administered BrdU in drinking water (1 mg/ml) 7 days prior to dissection. Tissue sections were also stained with hematoxylin and eosin to assess tumor pathology.

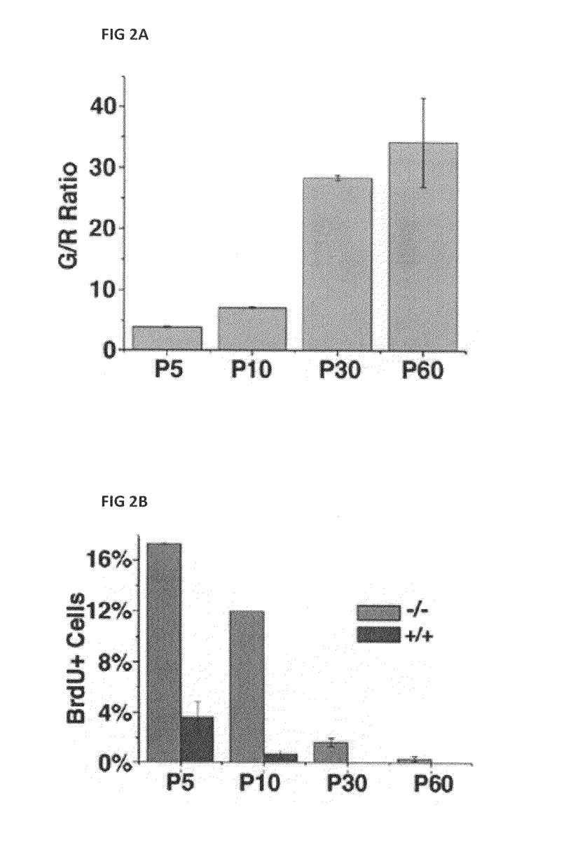

Because both green and red cells originated from the same progenitor, the ratio of cell numbers between green and red cells (referred to as G/R ratio hereafter) could serve as an indicator for the extent of over-expansion. Green and red cell numbers were counted in several representative brain regions including cerebral cortex, corpus callosum, striatum, thalamus, and hypothalamus, and G/R ratios were calculated. The average G/R ratio from P5 brain was around 4 and the value gradually increased to more than 30 as mice aged, indicating the manifestation of continuous growth advantage of mutant cells at early postnatal stages (FIG. 2A). As a control, the G/R ratio from the MADM-WT brain was quantified at the age of P60, where both red and green cells are WT in genotype. As expected, the G/R ratio in these mice was not significantly different from 1:1.

Next, the proliferative activity of these mutant cells was investigated by BrdU labeling to determine whether mutant cells have augmented proliferative activities as an early sign of tumorigenesis. Because of the short labeling time (1.5 hours), it was expected that only fast dividing cells can have time to incorporate BrdU into their genomic DNA. The quantification results showed that from P5 to P60, the BrdU+ cells in the whole brain decreased dramatically (FIG. 2B). BrdU incorporation among three genotypes of cells (null, wild type, or heterozygous) based on their MADM labeling was also investigated. The mutant cells gradually dominated the whole BrdU+population during brain development (from 25.9% at P5 to 100% at P60), while WT cells were rarely labeled by BrdU. At any time point examined, the percentage of BrdU+ cell among the mutant cell population was significantly higher than that of WT population in the same brain, although the percentage of BrdU+ cells decreased dramatically in both populations over time (FIG. 2B). All these data strongly suggest that the cell-cycle exit is delayed in the mutant population compared to the cells with the other two genotypes, and also that the mutant cells have more potential to proliferate compared with their WT counterparts.

At post-natal day 10, homozygous mutant p53 and NF1 (green) cells were dispersed throughout the brain. At this time point, a significant number of cells were dividing, as identified with Ki67 staining. Among the cells that were dividing, green (mutant) cells substantially outnumbered red (wild type) cells. At day P21, green cells outnumbered red cells by more than 100-fold and all observed dividing cells (80/80) were mutant (green) cells. At age 2 months, hyperplasia of green (mutant) cells was observed throughout the brain, with enrichment in distinct regions, particularly the white matter. At this time point, the green cells outnumbered the red (wild type) cells by more than 100-fold. However, staining with the cell division marker Ki67 indicated that these cells were not actively dividing. Further, there was no sign of tumor pathology in the brains examined.

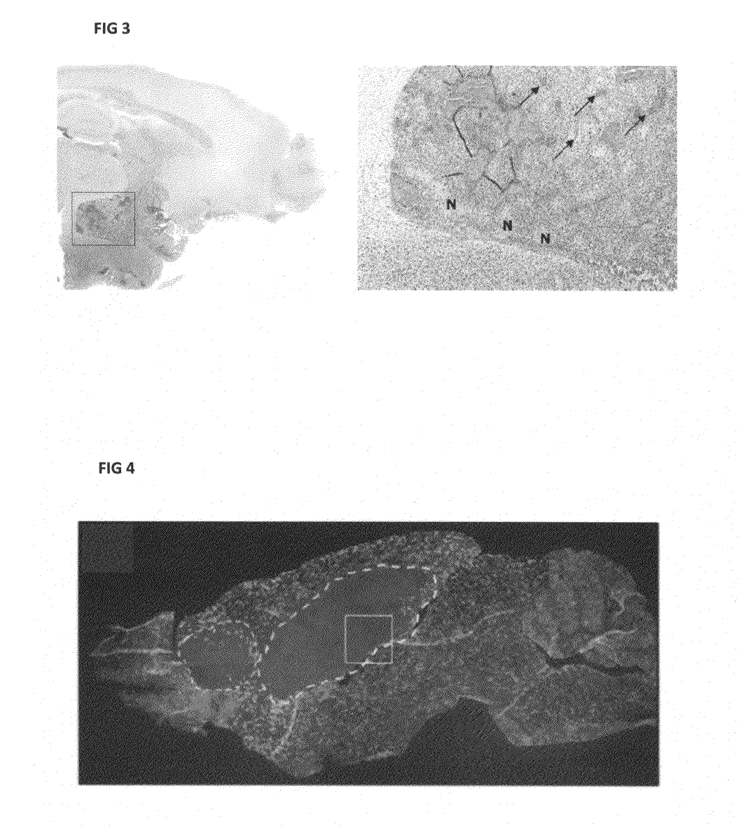

At age 4-5 months, all mice analyzed (7/7) had developed malignant gliomas (grade III and IV) based on pathological features, including pseudopalisading necrosis and prominent blood vessels (FIG. 3), as well as peri-neuronal satellitosis. The tumor location and latency for the mice is shown in Table 3. The tumors consisted mostly of green (mutant) cells (FIG. 4), indicating that they were homozygous for the p53 and NF1 knockout alleles. Dividing cells were identified using Ki67 staining. All dividing cells in the tumor mass were green, although not all green cells were dividing.

TABLE 3
Glioma latency and location in p53-NF1 MADM mice

| Mouse ID | Age | | Tumor(s) in brain |
|---|---|---|---|
| 6290 | 4.5 | months | Ventral forebrain |
| | | | Midbrain |
| 6291 | 4 | months | Neocortex |
| | | | Brain stem |
| 7436 | 4 | months | Corpus callosum |
| 7583 | 4.5 | months | Highly diffuse tumor |
| 6256 | 4.5 | months | Neocortex |
| | | | Brain stem |
| 5086 | 4.5 | months | Near olfactory bulb (small) |
| 5583 | 5 | months | Forebrain (large) |

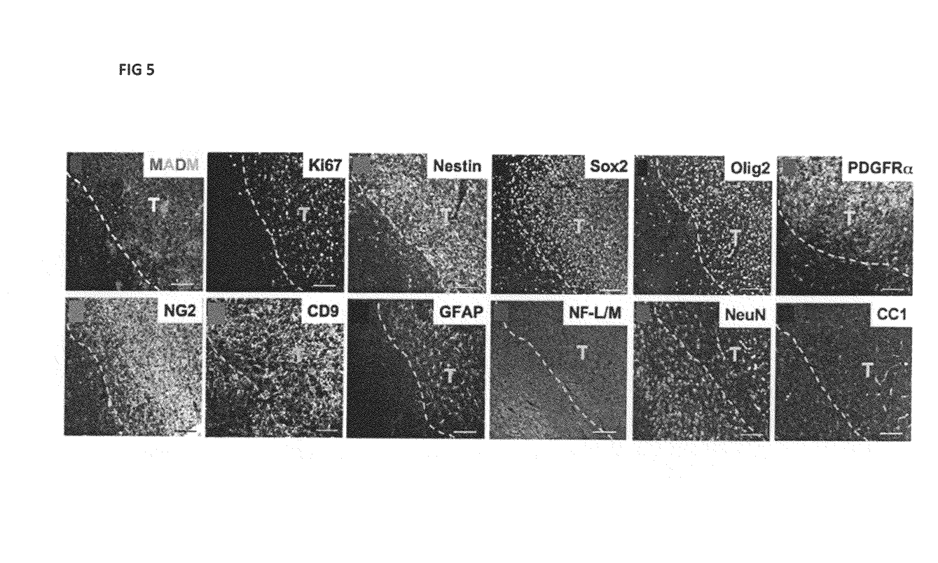

In addition to the pathological features, many classic glioma makers (such as Nestin, Sox2, Olig2 and PDGFRα) were found to be highly expressed in tumor regions, while mature cell type markers such as Neurofilament and NeuN for neurons, CC1 for oligodendrocytes were absent (FIG. 5).

### Example 4

### Glioma Cell of Origin

This example describes characterization of the cell of origin of gliomas in the p53-NF1 MADM mice.

It has been suggested that malignant glioma arises from astrocytes because of strong staining of gliomas for GFAP, a marker for mature astrocytes (Bachoo et al., Cancer Cell 1:269-277, 2002). Another hypothesis is that gliomas arise from neural stem cells in the subventricular zone of adult brain, because gliomas often stain for almost all types of neuronal markers, ranging from glial precursors to mature glial cells. However, such heterogeneous cell types could also be normal cells mixed in the tumor mass.

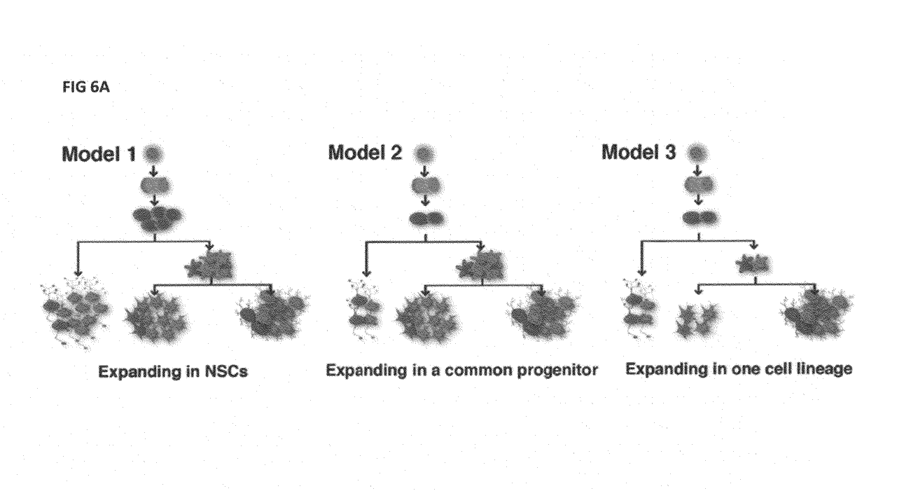

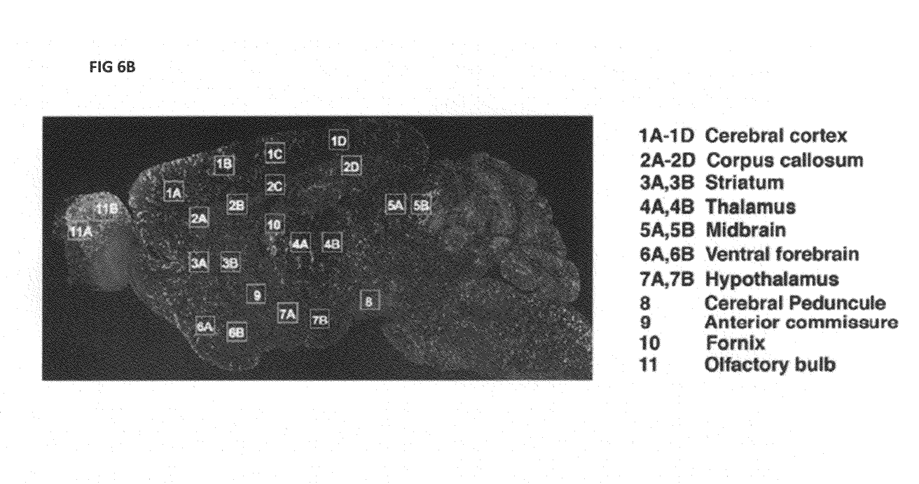

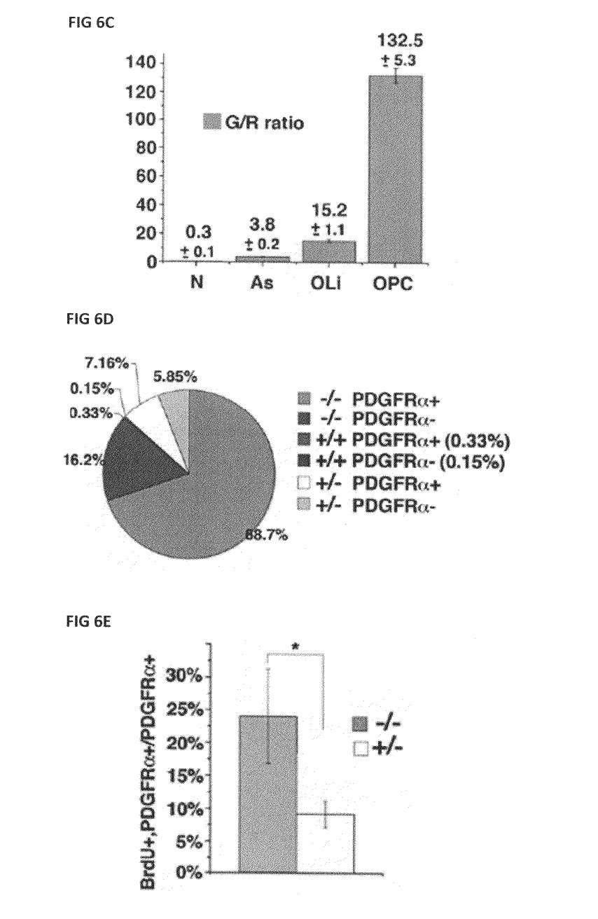

As the first mutation was introduced into embryonic neural stem cells (NSCs) using the NSC-specific Cre lines (nestin-Cre or hGFAP-CRE; Example 2), it was expected that the mutant cells in the adult brains would consist of a heterogeneous population. Because of the permanent labeling of all progeny cells from embryonic NSCs by the MADM system, it was predicted that if the major overexpansion of the mutant cells happened at the early embryonic NSCs, it would result in a similar level of overexpansion, hence a similar G/R ratio, of all cell types in the adult brain (as in FIG. 6A, model 1). Alternatively, if the over-expansion happened in a progenitor of more than one cell type, the G/R ratio would be higher in all the progeny of that progenitor (FIG. 6A, model 2). Or, if the over-expansion occurred in a specific cell lineage, the G/R ratio of that lineage would be significantly higher in some cell types than in others (FIG. 6A, model 3). The quantification from 16-21 distinct brain regions, which include the most typical forebrain structures (FIG. 6B), revealed that in 2-month old MADM pre-malignant mouse brain the G/R ratios of four cell types (neuron, astrocyte, oligodendrocyte, and OPC) were not equal (FIG. 6C). In contrast to no or very limited overexpansion of mutant neurons, astrocytes and oligodendrocytes, mutant OPCs showed more than 100-fold increase over WT OPCs. Therefore, by tracking the progression of tumorigenesis, it was found that, although the gene mutations initiated in NSCs, major overexpansion only occurred within the OPC lineage.

Although mutant OPCs showed drastic overexpansion at pre-malignant stages, it is conceivable that they must have increased proliferative activity to further progress into malignant tumors. Short-term BrdU labeling, however, showed that the proliferation of mutant cells was very infrequent in the adult brain (only accounted for 0.3%±0.2% of all examined mutant cells at P60). Mutant cell types may divide slowly prior to full malignancy, which would evade detection by the short-term BrdU labeling approach. Therefore, the BrdU labeling time was increased to one week to identify all dividing cells irrespective to their cell cycle rate. Twenty-one brain regions in both gray and white matter were analyzed. From three P60 MADM-mutant brains 1704 BrdU+ cells in total were identified, among which 1468 were mutant (accounting for 86.2% of the BrdU labeled population) but much fewer were heterozygous and only 10 were red WT cells. These data, together with those in Example 3 indicate that the mutant cells were the major proliferative pool.

Next, the identity of BrdU+ cell type(s) was examined with a four-channel immuno-fluorescent co-staining method (MADM takes green and red channels, while cell identity marker and BrdU occupy blue and far-red channels). Again it was found that OPCs were the major cell type labeled by BrdU. Co-staining with PDGFRα showed that 100% of all BrdU+ mutant cells in short-term (1.5 hours) BrdU labeled brains and more than 80% of all BrdU+mutant cells in 7-day long-term labeled brains were PDGFRα+ (FIG. 6D). The remaining ˜20% BrdU+PDGFRα-mutant cells were differentiated oligodendrocytes, based on marker staining and their distinct morphologies revealed by the resolution of MADM-labeled cells. Therefore, most, if not all, proliferating mutant cells in brain parenchyma were OPCs or their progeny. Since this phenomenon could be explained by the overwhelming abundance of mutant cells rather than their augmented proliferative activity, the percentage of BrdU+ cells in distinct populations (mutant vs. heterozygous cells) was calculated. Consistent with previous reports that normal OPCs can divide slowly in adult rodent brains, it was found that ˜10% heterozygous OPCs were labeled by BrdU. However, this value was much lower than % BrdU labeling in mutant OPCs (FIG. 6E), suggesting that mutant OPCs have a larger proliferative pool than their heterozygous counterpart. The rarity of MADM-WT OPCs precluded the precise quantification of the BrdU labeling efficiency in that population.

The above described quantification did not include subventricular zone (SVZ), where slow-dividing adult neural stem cells reside (Doetsch et al., Cell 97:703-716, 1999). Due to the densely packed arrangement of adult NSCs, all BrdU+ cells in the SVZ were quantified, including type B NSCs and their progeny (type C and A cells) (Doetsch et al., Cell 97:703-716, 1999). Among all 5705 BrdU+ cells quantified, only 1.1% were green and the G/R ratio was not significantly different than 1 (0.92±0.11, P=0.52), suggesting that the mutant NSC population had no detectable overexpansion. This finding was further strengthened by the lack of detectable expansion of mutant interneuron population in olfactory bulbs, which consists of main population of progeny from adult SVZ NSCs. To further investigate the expansion of NSC population, the G/R ratio of germinal zones in embryonic brains at E15.5 was quantified. The average G/R ratio at either dorsal or ventral stem cell zone was close to 1. Strikingly, even when cells in the entire brain were counted, there was no overexpansion of mutant cells, correlating well with the lack of MADM-labeled PDGFRα+OPCs at this stage.

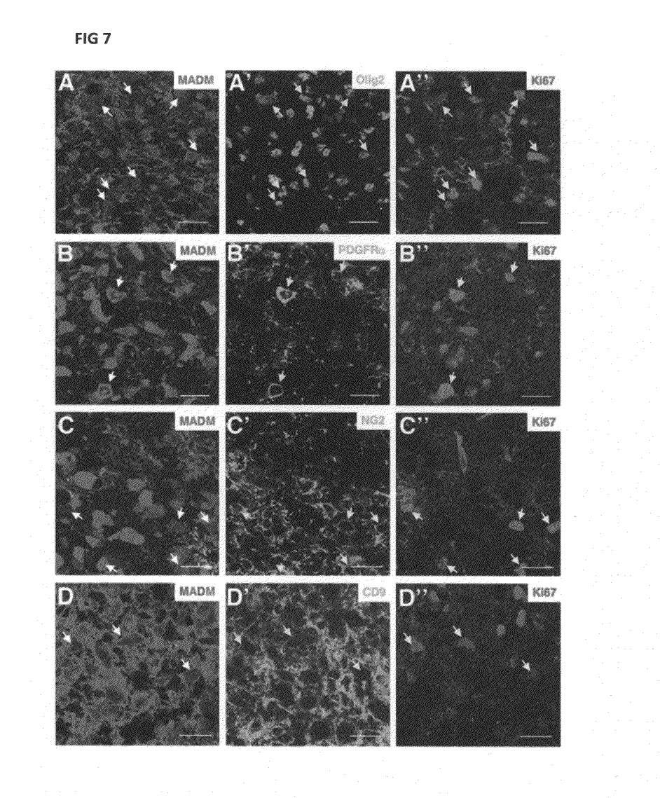

Tumor mass often consists of both mutant cells and engulfed WT cells, and it is of great interest to tell them apart. Unambiguous labeling of mutant cells by MADM provides such a resolution to investigate whether OPC markers are expressed in tumor cells per se. Confocal images with high magnification revealed that dividing mutant cells (GFP+Ki67+) in all gliomas strongly expressed a panel of OPC-specific markers, including Olig2, PDGFRα, NG2 and CD9 (FIG. 7). In some tumor samples tumor cells also expressed Nestin, although the expression was highly variable among different samples. Green tumor cells did not express mature cell markers such as GFAP, NeuN, or CC1. GFAP+ cells in the tumor were neither green nor Ki67+, consistent with the original report that they are likely recruited reactive astrocytes in this mouse model (Reilly et al., Nat. Genet. 26:109-113, 2000.

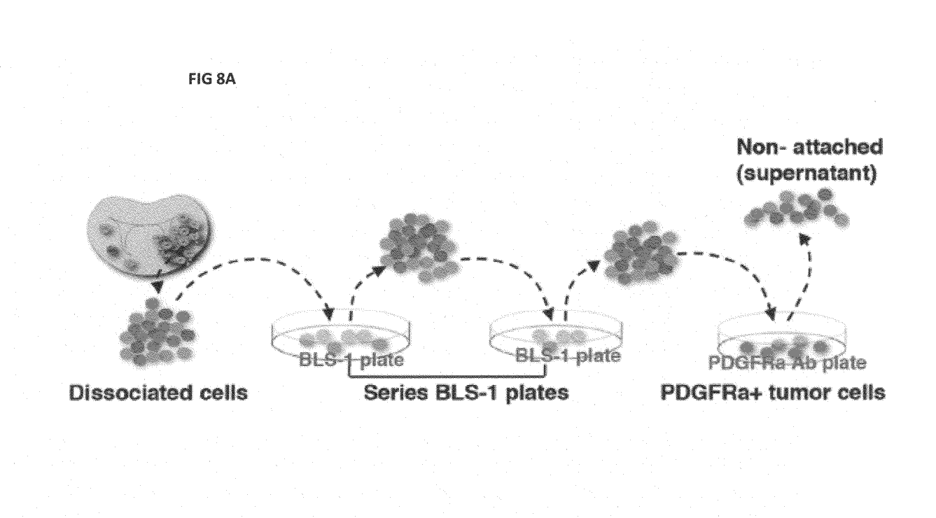

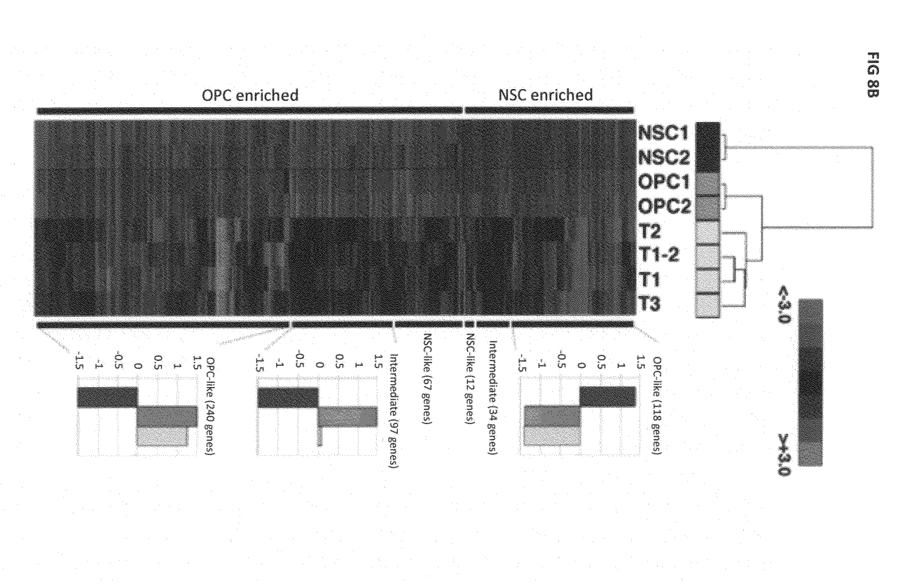

To further characterize the nature of these tumor cells, they were enriched based on their surface expression of PDGFRα with the immuno-panning method that is widely used for the purification of normal OPCs (FIG. 8A and Cahoy et al., J. Neurosci. 28:264-278, 2008; Barres et al., Cell 70:31-46, 1992). Briefly, dissociated cells were incubated in a BLS-1 coated plate to remove microglial cells, then the supernatant was moved onto a plate coated with anti-PDGFRα antibody that selectively binds to PDGFRα+OPCs but not other cell types. Realtime qRT-PCR showed a significantly higher level of PDGFRα mRNA expression in the immuno-panning enriched cells (abbreviated as panned tumor cells hereafter) than in the supernatant fraction, demonstrating the success of this method. More than 99% of the panned tumor cells also expressed NG2. The panned tumor cells also expressed Olig2 and CD9, consistent with the in vivo observations discussed above. A significant number of these purified tumor cells (up to more than 90% in some tumors) also expressed O4, a functional marker specifically expressed by adult OPCs and pre-oligodendrocytes (Nishiyama et al., Nat. Rev. Neurosci. 10:9-22, 2009). However, no purified tumor cells were found to express mature oligodendrocyte markers, such as O1 and MBP.

Although both in vivo and in vitro marker staining implicated the OPC nature of these glioma cells, it would be most informative to analyze the global gene expression profile. The whole transcriptome expression profile of the panned tumor cells was compared to that of purified WT neonatal (P8) OPCs. Neurospheres derived from E14.5 embryo forebrains (enriched in NSCs) were included as a control, to investigate whether these tumor cells have stem cell signatures. Total RNA was extracted from above mentioned cells and labeled with Cy3 dye, and total RNA from P17 wildtype brains was extracted and labeled with Cy5 dye as a universal reference to allow the comparison between tumor cells, OPCs and NSCs. To compare the similarities between tumor cells and OPCs vs. NSCs, a list of 568 genes with at least a 4-fold difference in expression between NSCs and OPCs was compiled (FIG. 8B). Then the dendrogram derived from the clustering analysis was used to evaluate the similarity between each cell type. Of all 568 genes that were differentially expressed between NSCs and OPCs, tumor cells share the expression profile with OPCs for over 63% of them. Furthermore highly-expressed genes in both WT OPCs and tumor cells included many known OPC genes such as PDGFRα, NG2 (CSPG4), Sox10, CD9, Hes5, and Sox6 (Cahoy et al., J. Neurosci. 28:264-278, 2008). The expression of some of these genes in multiple tumor cells was verified by realtime qPCR. They all had comparable expression levels to WT OPCs, while embryonic neurospheres had very low/no expression.

Single sample GSEA (Gene Set Enrichment Analysis) activation scores of neural lineage gene sets revealed the close resemblance of glioma cells to OPCs but not other cell types (FIG. 8C). Gene sets of 250 up- and down-regulated marker genes in all brain cell types were derived from Cahoy et al. (supra) and projected onto the glioma samples as described previously (Barbie et al., Nature 462:108-112, 2009; Verhaak et al., Cancer Cell 17:98-110, 2010). After comparing gene expression profiles of the purified tumor samples with the published dataset for all four distinct cell types, only the transcriptome of OPC showed significant similarity to that of the tumor cells (FIG. 6D). These data further strengthen the finding that glioma cells in the p53-NF1 MADM mouse model are closely related to OPCs but not other cell types in the brain.

It is also important to note that, although tumor cells showed an OPC-like expression profile, 14% of genes in the microarray analysis actually shared similarity to NSCs rather than to OPCs. Vimentin was among the 12 upregulated genes in both NSC and tumor cells, which is a known marker for both NSCs and human glioma samples (Yung et al., J. Neurooncol. 3:35-38, 1985). Consistent with the in vivo staining data, the commonly used NSC marker Nestin showed highly variable expression levels among different tumor samples.

Since astrocytes had long been suspected to be involved in glioblastoma multiforme (GBM) while OPCs were less known, it was investigated to determine whether the p53-NF1 MADM model is relevant to human GBMs. Recently, the Cancer Genome Atlas (TCGA) project classified human GBMs into four distinct subtypes based on their gene expression profiles (Verhaak et al., Cancer Cell 17:98-110, 2010). To investigate whether the p53-NF1 MADM tumor model mimics any subtype of human GBMs, the microarray data of the purified tumor cells was compared with those of four human GBM subtypes classified based on 173 core human glioma samples. The comparison clearly showed that the p53-NF1 MADM mouse model closely resembled the proneural subtype of human GBMs (FIG. 8D). Expression features of the proneural subtype are reminiscent of oligodendrocytes but not the astrocyte lineage (Verhaak et al., Cancer Cell 17:98-110, 2010,) paralleling the findings that purified tumor cells in the p53-NF1 model are OPC-like. Therefore the commonality of the molecular features between OPC-like tumor cells and human glioma subtype highlights the relevance of this mouse model to human GBMs.

### Example 5

### Inducible p53-NF1 MADM Mouse

This example describes characterization of a tetracycline-inducible p53-NF1 MADM mouse.

To further investigate the initiation of glioma development, a doxycycline (Dox)-inducible Cre system (CMV-actin-rtTA/TRE-Cre) was introduced into the p53-NF1 MADM system. GT mice were crossed with CMV-actin-rtTA/TRE-Cre mice (TRE-Cre mice were obtained from Jackson Laboratory (Bar Harbor, Me.; Cat. No. 006224) and rtTA mice were as described in Sarin et al., Nature 436:1048, 2005). The resulting mice were crossed with TG, p53KO, NF1FLOX mice (described in Example 2). Cre expression was induced by a single intraperitoneal Dox injection (50 μg/25 g body weight) in pregnant mice at embryonic day 8.5. Following birth, the mice were dissected at day P10 and examined for the presence of green (mutant) cells and expression of various neuronal cell markers. The Dox-inducible MADM recombination generated well-isolated clones of mutant cells at day P10, with dramatic overexpansion of mutant (green cells) compared to wild type (red) cells. Mutant cells expressed PDGFRα, indicating that they were halted at the polydendrocyte stage. In contrast, wild type cells expressed the mature oligodendrocyte marker CC1. Most dividing cells were mutant (green) cells, while most wild type (red) cells were not dividing, as assessed by Ki67 expression.

### Example 6

### Purification and Characterization of Tumor Cells

This example describes purification of tumor cells from p53-NF1 MADM mice and their characteristics in culture.

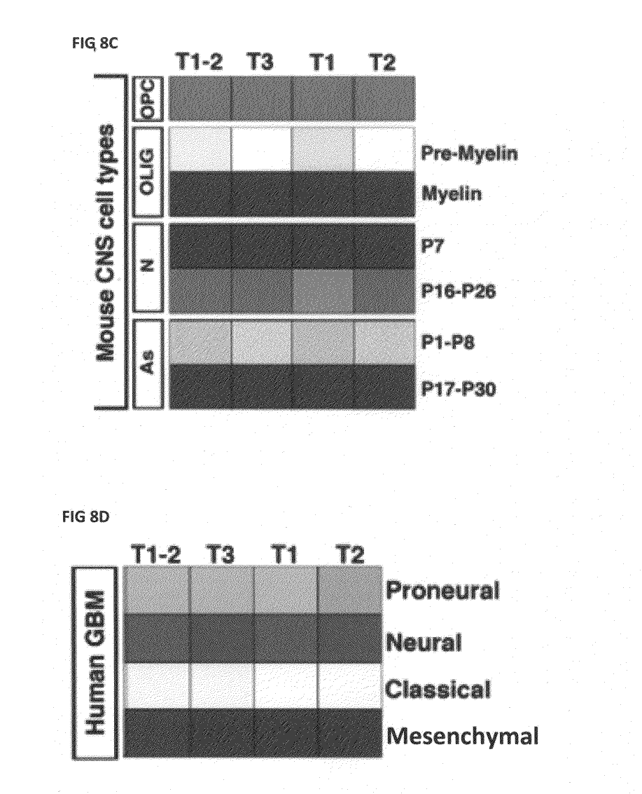

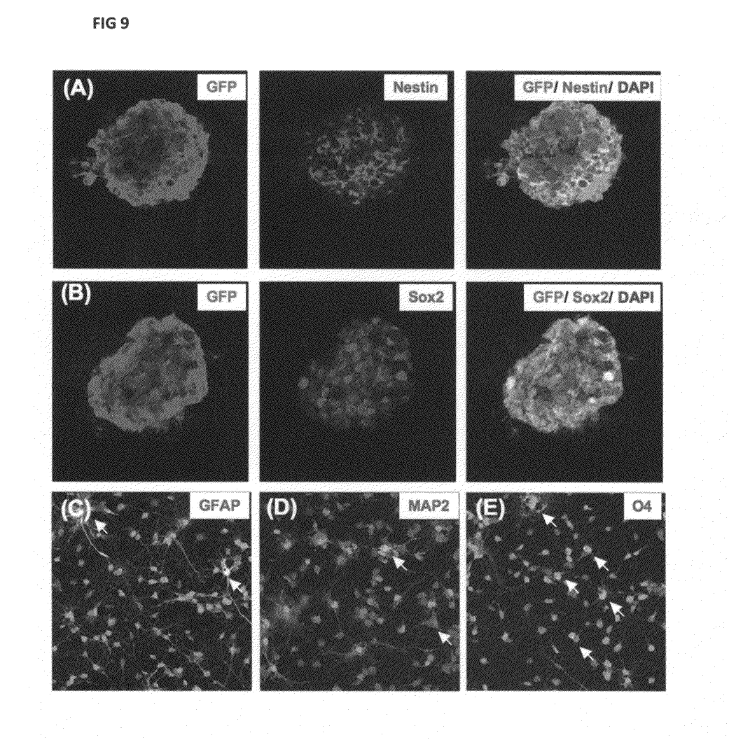

The center of a glioma that developed in an approximately 5 month old p53-NF1 MADM mouse was dissected and Papain reagents from Worthington Biochemical Corporation were used to dissociate the tumor mass into individual cells. The cells were grown in Neurobasal medium with B27 supplement (Invitrogen, Carlsbad, Calif.) containing 20 ng/ml EGF and 20 ng/ml FGF (serum-free). Green “tumor-spheres” formed after 5-7 days in culture (FIGS. 9A and B). These spheres showed three aspects of stem cells. They stained positive for stem cell markers Nestin and Sox2 (FIGS. 9A and B); they could self-renew and form secondary spheres; and they could differentiate into astrocytes (FIG. 9C), neurons (FIG. 9D), and oligodendrocytes (FIG. 9E) when exposed to 1% serum for 3-5 days.

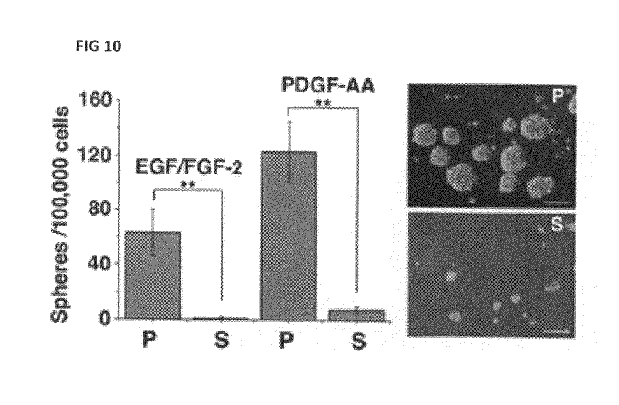

Tumor sphere formation ability was tested by culturing the OPC-like tumor cells obtained by panning (Example 4) in NSC media, with EGF and FGF-2 as mitogens. Similar to WT NSCs, all panned tumor cells tested could efficiently form tumor spheres after being seeded at clonal density (FIG. 10). The sphere forming rate (percentage of sphere number per 100,000 seeded cells) ranged from 1% to 5% among different samples. These spheres expressed both NSC markers and OPC markers and could be passaged repeatedly, demonstrating their self-renewal ability in vitro. Furthermore, the supernatant fraction from the panning plate (with OPC-like tumor cells depleted) could rarely form tumor spheres, confirming the enrichment of tumor propagating cells in the panned fraction. It was reported that PDGFRα is also expressed by adult NSCs (Jackson et al., Neuron 51:187-199, 2006), which could be a source for sphere formation in these assays. However, only 0.2% of the population in panned tumor cells was PDGFRα+NG2-(possible adult NSCs). Compared to the 1-5% tumor sphere-forming rate, this suggests that most tumor spheres, if not all, come from the NG2+OPC-like tumor cells (non-NSC cells). Also, all tested OPC-like tumor cells had the ability to grow into tumor spheres with PDGF-AA as the sole growth factor, which is an essential mitogen for WT OPCs but should not support the growth of NSCs.

Next the multi-potentiality of panned tumor cells was examined. Under the appropriate conditions, these OPC-like tumor spheres gave rise to GFAP+astrocytes, O1+ oligodendrocytes and NeuN+ neurons, suggesting they have potential to differentiate into both glia and neurons.

A major feature of tumor propagating cells is their ability to recapitulate tumors in a host animal. To test this feature, either cultured or freshly panned tumor cells were injected into the brain of NOD-SCID mice. Four out of five tumor cell lines initiated new tumor formation within 4-6 weeks of transplantation, while the control group of mice injected with the same number of normal embryonic NSCs (N=5) did not form any detectable tumors. To test the in vivo renewability of tumor propagating capacity of OPC-like tumor cells, panned tumor cells from secondary tumors (two independent cell lines) were injected into new NOD-SCID mice. Tertiary tumors formed within 4 weeks for both lines tested. The secondary and tertiary tumors recapitulated the features of primary tumors.

In addition to pathological resemblance, gene expression profiles between primary and secondary tumors were compared with microarray analysis. The clustering analysis showed that the secondary tumor closely resembled its original primary tumor. Realtime qPCR data also confirmed the comparable expression of all OPC-specific genes between primary and secondary tumors. These data demonstrated that secondary tumors faithfully recapitulated primary tumors at both the pathological and the molecular level.

In summary, these data show that the OPC-like tumors cells can function as tumor propagating cells, based on their capability to form tumor spheres and exhibit broad differentiation potential in vitro, and to recapitulate the gliomas in vivo in serial transplantation assays.

### Example 7

### Cell-Based Methods for Identifying Compounds for Treating or Preventing a Tumor

This example describes representative cell-based methods for screening compounds using cells from the transgenic mice disclosed herein to identify compounds that are useful for treating or preventing a tumor. However, one skilled in the art will appreciate that methods that deviate from these specific methods can also be used to successfully identify compounds that are useful for treating or preventing a tumor.

One or more test compounds (or compounds identified as a candidate in previous assays) are obtained and screened for their effect on at least one phenotype of cells isolated from the transgenic p53-NF1 MADM, p53 MADM, or NF1 MADM mice disclosed herein.

Cells that are homozygous null for p53 and NF1 and that express GFP (mutant, green cells) are purified from transgenic mice described herein. For example, tissue (for example brain tissue, such as a glioma) that includes one or more cells that are homozygous null for p53 and NF1 and that express GFP are isolated from a p53-NF1 MADM mouse, dissociated and the green cells are isolated using fluorescence activated cell sorting (FACS). Cells that are wild type for p53 and NF1 and that express tdT (wild type, red cells) are separately purified using FACS from tissue (for example brain tissue, such as a glioma) that includes one or more cells that are wild type for p53 and NF1 and that express tdT isolated from a p53-NF1 MADM mouse. The purified cells are placed in a cell culture at a starting ratio of 1:1 of mutant (green) cells to wild type (red) cells. The cell culture is contacted with at least one test compound, such as 1, 2, 3, 4, 5, 6, 7, 8, 9, 10, or more test compounds (dosage ranging from 1 nM to 1 mM) for about 1 day to about 1 week. The number of green cells in the culture is determined by fluorescence microscopy or FACS. The number of red cells in the culture may also be determined by fluorescence microscopy or FACS and a ratio of green cells to red cells determined. Similar methods may be used with the p53 MADM mice or NF1 MADM mice disclosed herein.

Contacting the culture with at least one test compound that is a good candidate for treating or preventing a tumor is expected to decrease the number of mutant (green) cells in the culture when compared to untreated control cells. A decrease in the ratio of mutant (green) to wild type (red) cells is also expected in cells contacted with at least one test compound that is a good candidate for treating or preventing a tumor as compared to untreated control cells.

### Example 8

### In Vivo Methods for Identifying Compounds for Treating or Preventing a Tumor

This example describes representative methods for screening compounds using the p53-NF1 MADM transgenic mice, p53 MADM transgenic mice, or NF1 MADM transgenic mice disclosed herein to identify compounds that are useful for treating or preventing a tumor. However, one skilled in the art will appreciate that methods that deviate from these specific methods can also be used to successfully identify compounds that are useful for treating or preventing a tumor.

One or more test compounds (or compounds identified as a candidate in previous assays) are obtained and screened for their effect on at least one phenotype of the transgenic p53-NF1 MADM mice, p53 MADM mice, or NF1 MADM disclosed herein. The transgenic mice are treated with at least one test compound, such as 1, 2, 3, 4, 5, 6, 7, 8, 9, 10, or more test compounds (dosage ranging from 0.1 pg/kg to 10 mg/kg). In one example, the test compound is provided by parenteral administration (such as intraperitoneal injection) or by oral administration. The test compounds are administered to the transgenic mice (for example, every 12 hours or every 24 hours (daily)) for an appropriate period of time (such as 1 week, 2 weeks, 3 weeks, one month, two months, three months, six months, or more). The test compounds may be administered prior to the expected development of a glioma (for example, beginning at birth or about 2 weeks, one month, two months, or three months post-natal), at the time of expected development of a glioma (for example, beginning about four months post-natal), or following the development of glioma (for example, beginning five months, six months, or more post-natal). Untreated animals are used as controls. Control animals are administered vehicle via the appropriate administration method on the same dosing schedule as the animals treated with one or more test compounds.

Animal tissue is subjected to gross tissue morphology and histology studies, especially focused on the brain. Gross tumor morphology and histological hallmarks of glioma are assessed. The number and size of tumors in the treated animals is compared to control animals. The number of mutant cells (those homozygous for p53 and NF1 knockout alleles, those homozygous for p53 knockout alleles, or those homozygous for NF1 knockout alleles, for example, those expressing GFP) is measured in a tissue sample, for example using fluorescence microscopy. In some examples, the number of cells that are wild type for p53 and NF1, wild type for p53, or wild type for NF1 (for example, those expressing tdT) is also measured and a ratio of mutant to wild type cells is determined. Immunohistochemical analysis is conducted on fixed tissue samples to visualize known tumor markers, such as GFAP, Sox2, NG2, PDGFRα, Nestin, and Olig2 in the case of a glioma, as well as markers of cell proliferation (such as Ki67 expression or BrdU incorporation) in comparison to control samples.

Administration of a test compound that is a good candidate for treating or preventing a tumor is expected to decrease the size or number of tumors when compared to untreated control mice. A decrease in the number of cells homozygous null for p53 and NF1, homozygous null for p53, or homozygous null for NF1, or a decrease in the ratio of mutant to wild type cells, is also expected in mice administered a test compound that is a good candidate for treating or preventing a tumor as compared to untreated control mice. Administration of a test compound that is a good candidate for treating or preventing a tumor is also expected to decrease expression of tumor markers and markers of cell proliferation when compared to untreated control mice.

In view of the many possible embodiments to which the principles of the disclosure may be applied, it should be recognized that the illustrated embodiments are only examples and should not be taken as limiting the scope of the disclosure. Rather, the scope of the invention is defined by the following claims. We therefore claim as our invention all that comes within the scope and spirit of these claims.

We claim:

1. A transgenic mouse the genome of which comprises:
a) a first nucleic acid molecule comprising a first promoter operably linked to a nucleic acid molecule encoding an N-terminal portion of a green fluorescent protein (GFP) and a C-terminal portion of a tdTomato (tdT) fluorescent protein, wherein the N-terminal portion of the GFP and the C-terminal portion of the tdT protein are separated by a β-globin intron comprising a first loxP site, and wherein the first nucleic acid molecule is present at a locus of a first chromosome 11 of a chromosome 11 pair;
b) a second nucleic acid molecule comprising a second promoter operably linked to a nucleic acid molecule encoding an N-terminal portion of the tdT protein and a C-terminal portion of the GFP, wherein the N-terminal portion of the tdT protein and the C-terminal portion of the GFP are separated by the β-globin intron comprising a second loxP site, and wherein the second nucleic acid molecule is present at the homologous locus of the second chromosome 11 of the chromosome 11 pair;
c) a mutated gene selected from the group consisting of a heterozygous null mutation in the p53 gene on the second chromosome 11 of the chromosome 11 pair, a heterozygous floxed neurofibromatosis type 1 (NF1) gene on the second chromosome 11 of the chromosome 11 pair, and a combination thereof; and
d) a third nucleic acid molecule encoding a Cre recombinase operably linked to a third promoter, wherein Cre recombinase-promoted somatic recombination between the first and second loxP sites occurs in at least one cell of the transgenic mouse resulting in at least a first cell comprising a first recombined nucleic acid molecule encoding a functional GFP and a homozygous null mutated gene and at least a second cell comprising a second recombined nucleic acid molecule encoding a functional tdT protein and a homozygous wild type mutated gene.

2. The transgenic mouse of claim 1, wherein the nucleic acid molecule encoding the Cre recombinase is operably linked to a human glial fibrillary acidic protein (GFAP) promoter.

3. The transgenic mouse of claim 1, wherein the locus of the first and second nucleic acid molecules on the chromosome 11 pair is between an Eif4enif1 gene and a Drg1 gene.

4. The transgenic mouse of claim 1, wherein the first promoter or the second promoter comprises a CMV (3-actin promoter.

5. The transgenic mouse of claim 1, wherein the transgenic mouse develops a glioma, wherein the glioma comprises at least one cell comprising the first recombined nucleic acid molecule encoding the functional GFP, the homozygous null mutation in the p53 gene, and the homozygous null mutation in the NF1 gene.

6. A method for identifying a compound for treating or preventing a tumor, comprising:
administering at least one test compound to the transgenic mouse of claim 1;
determining a phenotype of the transgenic mouse; and
selecting a test compound that decreases the phenotype of the transgenic mouse as compared to a control, thereby identifying a compound that treats or prevents the tumor.

7. The method of claim 6, wherein the phenotype of the transgenic mouse comprises a number of cells expressing GFP or a tumor characteristic.

8. The method of claim 7, wherein the tumor characteristic is selected from the group consisting of tumor size, tumor cell gene expression, tumor growth, tumor number, tumor metastasis, and tumor recurrence.

9. A method for identifying a compound for treating or preventing a tumor, comprising:
culturing in vitro at least one first cell from the transgenic mouse of claim 1;
contacting the first cell with at least one test compound;
determining a phenotype of the first cell; and
selecting a test compound that alters the phenotype of the first cell as compared to a control, thereby identifying a compound that treats or prevents the tumor.

10. The method of claim 9, further comprising:
co-culturing in vitro the at least one first cell with at least one second cell from the transgenic mouse of claim 1.

11. The method of claim 9, wherein the phenotype comprises cell number, cell proliferation, cell cycle stage, cell death, cell morphology, cell gene expression, or presence of one or more tumor sphere characteristic.

12. The method of claim 11, wherein the selected test compound decreases cell number, cell proliferation, or gene expression of the first cell as compared to the control.

13. The method of claim 11, wherein the selected test compound increases cell death of the first cell as compared to the control.

14. The method of claim 10, wherein the at least one first cell and at least one second cell are initially present in the in vitro culture at a 1:1 ratio.

15. The method of claim 14, wherein the phenotype is the ratio of the at least one first cell to the at least one second cell.

16. The method of claim 15, wherein the selected compound decreases the ratio of the at least one first cell to the at least one second cell.

17. A transgenic mouse the genome of which comprises:
a) a nucleic acid molecule comprising a promoter operably linked to a nucleic acid molecule encoding an N-terminal portion of a tdTomato (tdT) fluorescent protein and a C-terminal portion of a green fluorescent protein (GFP), wherein the N-terminal portion of the tdT protein and the C-terminal portion of the GFP are separated by a β-globin intron comprising a first loxP site, and wherein the first nucleic acid molecule is present at a locus of a both chromosomes 11 of a chromosome 11 pair;
b) a heterozygous null mutation in the p53 gene on a first chromosome 11 of the chromosome 11 pair; and
c) a heterozygous floxed neurofibromatosis type 1 (NF1) gene on the first chromosome 11 of the chromosome 11 pair

18. A transgenic mouse the genome of which comprises a first nucleic acid molecule comprising a first promoter operably linked to a nucleic acid molecule encoding an N-terminal portion of a green fluorescent protein (GFP) and a C-terminal portion of a tdTomato (tdT) fluorescent protein, wherein the N-terminal portion of the GFP and the C-terminal portion of the tdT protein are separated by a β-globin intron comprising a first loxP site, and wherein the nucleic acid molecule is present at a locus i of both chromosomes 11 of a chromosome 11 pair.

19. The transgenic mouse of claim 18, further comprising a second nucleic acid molecule encoding a Cre recombinase operably linked to a promoter.

20. A transgenic mouse the genome of which comprises:
a) a nucleic acid molecule comprising a promoter operably linked to a nucleic acid molecule encoding an N-terminal portion of a green fluorescent protein (GFP) and a C-terminal portion of a tdTomato (tdT) fluorescent protein, wherein the N-terminal portion of the GFP and the C-terminal portion of the tdT protein are separated by a β-globin intron comprising a first loxP site, and wherein the first nucleic acid molecule is present at a locus of a both chromosomes 11 of a chromosome 11 pair;
b) a heterozygous null mutation in the p53 gene on a first chromosome 11 of the chromosome 11 pair; and
c) a heterozygous floxed neurofibromatosis type 1 (NF1) gene on the first chromosome 11 of the chromosome 11 pair

21. A transgenic mouse the genome of which comprises a first nucleic acid molecule comprising a first promoter operably linked to a nucleic acid molecule encoding an N-terminal portion of a tdTomato (tdT) fluorescent protein and a C-terminal portion of a green fluorescent protein (GFP), wherein the N-terminal portion of the tdT protein and the C-terminal portion of the GFP are separated by a β-globin intron comprising a first loxP site, and wherein the nucleic acid molecule is present at a locus of both chromosomes 11 of a chromosome 11 pair.

22. The transgenic mouse of claim 18, further comprising a second nucleic acid molecule encoding a Cre recombinase operably linked to a promoter.
